(19) United States
(12) Patent Application Publication
(10) Pub. No.: US 2013/0184243 A1
(43) Pub. Date: Jul. 18, 2013

(54) COMPOSITIONS OF ALKYLATING AGENTS AND METHODS OF TREATING SKIN DISORDERS THEREWITH

(71) Applicants: Robert Alonso, Wayne, PA (US); Martin Stogniew, Blue Bell, PA (US)

(72) Inventors: Robert Alonso, Wayne, PA (US); Martin Stogniew, Blue Bell, PA (US)

(73) Assignee: Ceptaris Therapeutics, Inc., Malvern, PA (US)

(21) Appl. No.: 13/546,149

(22) Filed: Jul. 11, 2012

Related U.S. Application Data
(60) Provisional application No. 61/537,153, filed on Sep. 21, 2011.
(60) Provisional application No. 61/506,222, filed on Jul. 11, 2011.

(51) Int. Cl. A61K 9/00; A61K 45/06; A61K 31/131

(52) U.S. Cl. CPC A61K 9/0014; A61K 31/131; A61K 45/06
USPC 514/171; 514/672; 514/569

(57) ABSTRACT

Provided are compositions comprising alkylating agents, including nitrogen mustards, that are suitable for topical use, and methods for treating skin disorders comprising topically administering the compositions.

### CROSS-REFERENCE TO RELATED APPLICATIONS

This application claims priority to U.S. Provisional Application Nos. 61/506,222, filed on Jul. 11, 2011 and 61/537,153, filed on Sep. 21, 2011, both of which are incorporated by reference herein for all purposes.

### FIELD OF THE INVENTION

The invention encompasses compositions comprising alkylating agents, including nitrogen mustards, that are suitable for topical use, and methods for treating skin disorders comprising topically administering the compositions.

### BACKGROUND OF THE INVENTION

Alkylating agents, such as nitrogen mustards, have been used in the pharmaceutical industry as anti-cancer drugs. For example, nitrogen mustards have been used to treat cutaneous T-cell lymphoma (CTCL), including mycosis fungoides (MF).

CTCL is a cancer of the white blood cells that primarily affects the skin and only secondarily affects other sites. The disease involves the uncontrolled proliferation of T-lymphocytes known as T-helper (CD4+) cells of the immune system. The proliferation of T-helper cells results in the penetration, or infiltration, of these abnormal cells into the epidermal layer of the skin. The skin reacts with slightly scaling lesions that itch, although the sites of greatest infiltration do not necessarily correspond to the sites of the lesions. The lesions are most often located on the trunk, but can be present on any part of the body. In the most common course of the disease, the patchy lesions progress to palpable plaques that are deeper red and have more defined edges. As the disease worsens, skin tumors develop that are often mushroom-shaped, hence the name mycosis fungoides. Finally, the cancer progresses to extracutanous involvement, often in the lymph nodes or the viscera.

CTCL is a rare disease, with an annual incidence of about 0.29 cases per 100,000 persons in the United States. It is about half as common in Eastern Europe. However, this discrepancy may be attributed to a differing physician awareness of the disease rather than a true difference in occurrence. In the United States, there are about 500-600 new cases a year and about 100-200 deaths. CTCL is usually seen in older adults; the median age at diagnosis is 55-60 years. It strikes twice as many men as women. The average life expectancy at diagnosis is 7-10 years, even without treatment.

Thus, there is a need in the art for compositions of alkylating agents, such as nitrogen mustards, that are suitable for topical use.

### SUMMARY OF THE INVENTION

In one embodiment, the invention encompasses topical compositions comprising: (a) an effective amount of bis-(2-chloroethyl) methylamine or a pharmaceutically acceptable salt or solvate thereof; and (b) a pharmaceutically acceptable excipient, wherein the pharmaceutically acceptable excipient is an alcohol, a ketone, a dimethyl polysiloxane, an ethylene glycol derivative, a polyoxylglyceride, a polar aprotic solvent, an alpha-hydroxycarboxylic acids or a salt thereof, a diester of a dibasic acid, a polyethoxylated fatty acid, a PEG-fatty acid diester, PEG-fatty acid mono-ester mixture or an all-ester mixture, a polyethylene glycol glycerol fatty acid ester, an alcohol-oil transesterification product, a polyglycerized fatty acid, a propylene glycol fatty acid ester, a mixture of a propylene glycol ester and a glycerol ester, a mono- or diglyceride, a sterol or a sterol derivative, a polyethylene glycol sorbitan fatty acid ester, a polyethylene glycol alkyl ether, a sugar ester, a polyethylene glycol alkyl phenol, a polyoxyethylene-polyoxypropylene block copolymer, a polyoxyethylene, a sorbitan fatty acid ester, a lower alcohol fatty acid ester, an ionic surfactant, a penetration enhancer, or a thickening agent.

In another embodiment, the invention encompasses a method for treating a skin disorder comprising topically applying to a subject in need thereof a topical composition described above.

### DETAILED DESCRIPTION OF THE INVENTION

The invention meets a need in the art by providing compositions of alkylating agents, such as nitrogen mustards, that are suitable for topical use, and methods of treatment therewith.

### I. DEFINITIONS

As used herein, unless otherwise defined, the term “stable,” when referring to a composition of an alkylating agent, means that at least about 80% of the alkylating agent is present in the composition (in other words less than about 20% of the alkylating agent has degraded) after storage. Alternatively, the term “stable” means that the composition contains less than about 20% by weight of degradation product of the alkylating agent after storage.

As used herein, unless otherwise defined, the term “pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance, and bioavailability.

As used herein, unless otherwise defined, a “nitrogen mustard prodrug” is a compound that can be metabolized in vivo (i.e., can undergo chemical conversion my metabolic processes) to generate the nitrogen mustard.

As used herein, unless otherwise defined, “topical administration” means applying a drug to a localized area of the body or to the surface of a body part.

As used herein, unless otherwise defined, the term “effective amount” when referring to an alkylating agent means an amount of alkylating agent that is effective to treat a skin disorder.

As used herein, unless otherwise defined, the term “ameliorate” when referring to skin irritation means to lessen pain and reduce skin irritation.

As used herein, unless otherwise defined, the term “room temperature” means a temperature within the range of 15° C. to 30° C.

As used herein, unless otherwise defined, the term “degradation product,” when referring to an alkylating agent, means a compound that can be formed by the degradation of the alkylating agent, for example, by reaction of the alkylating agent with a nucleophile to displace one or more of the functional groups of the alkylating agent.

As used herein, unless otherwise defined, the term “nitrogen mustard degradation product,” means a compound that can be formed by the degradation of a nitrogen mustard, for example, by reaction of the nitrogen mustard with a nucelophile to displace one or more of the terminal chlorides of the nitrogen mustard.

As used herein, unless otherwise defined, the term “response,” when used in connection with treatment of a skin disorder in a human patient, means that the human patient's CAILS after treatment is greater than or equal to 50% lower than the human patient's CAILS prior to treatment and/or the human patient's SWAT score after treatment is greater than or equal to 50% lower than the SWAT score prior to treatment.

As used herein, unless otherwise defined, the term “efficacy-evaluable” or (“EE”), when referring to a patient population enrolled in a drug study, means patients who have received the drug for a period of at least about six months.

As used herein, unless otherwise defined, the term “technically-evaluable” or (“TE”), when referring to a patient population enrolled in a drug study, means all patients who have received at least about two months.

As used herein, unless otherwise defined, the term “intent-to-treat” or (“ITT”), when referring to a patient population enrolled in a drug study, means all patients who have received at least one dose of the drug.

### II. COMPOSITIONS OF ALKYLATING AGENTS

In one embodiment, the invention encompasses a composition comprising at least one alkylating agent or a pharmaceutically acceptable salt, solvate, or prodrug thereof, and at least one pharmaceutically acceptable excipient.

In one embodiment, the composition is suitable for topical administration. In one embodiment, the composition is in the form of a paste, a dispersion, a suspension, a solution, a gel, a cream, an emulsion, a foam, a lotion, or an ointment. In another embodiment, the composition is in the form of a dispersion. In another embodiment, the dispersion is a coarse dispersion, a colloidal dispersion, or a molecular dispersion.

A. Alkylating Agents

Suitable alkylating agents include, but are not limited to, a nitrogen mustard, a sulfur mustard, a Lewisite, an alkyl sulfonate, an ethyleneimine, a nitrosourea, a triazene, an imidazotetrazine, mechlorethamine, chlorambucil, cyclophosphamide, 4-hydroxycyclophosphamide, aldophosphamide, ifosfamide, melphalan, bis-(2-chloroethyl) ethylamine, tris-(2-chloroethyl)ethylamine, carmustine, fotemustine, lomustine, streptozocin, busulfan, dacarbazine, procarbazine, temozolomide, treosulfan, uramustine, hexamethylmelamine, thiotepa (N,N′,N″-triethylenethiophosphoramide), and tepa (N,N′,N″-triethylenephosphoramide), and pharmaceutically acceptable salts, solvates, and prodrugs thereof.

In one embodiment, the alkylating agent is a nitrogen mustard. In one embodiment, the nitrogen mustard is a compound of the following Structure (VII), (VIII), (IX), (X), (XII), (XIII), (XIV), (XV), (XVI), (XVII), (XVIII), or (XIX):

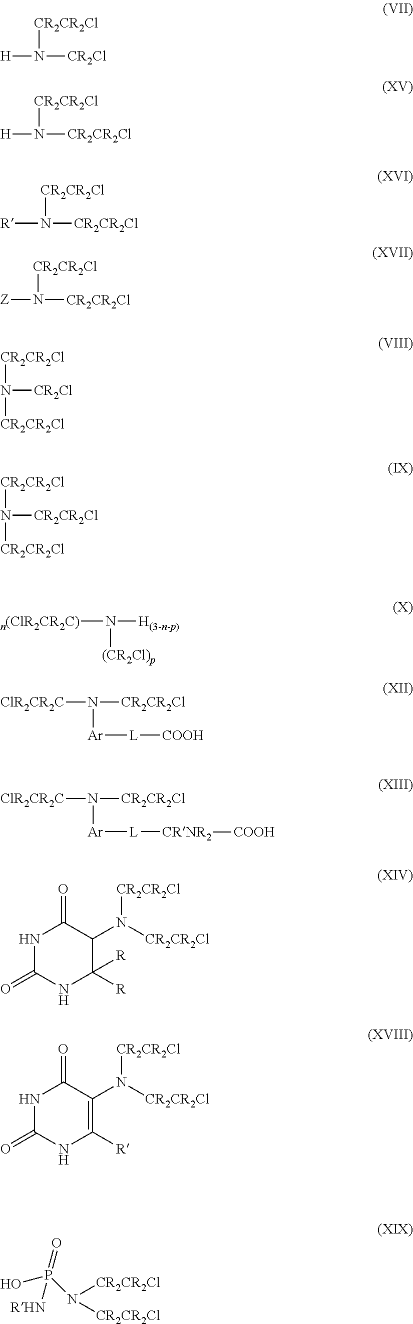

wherein:

each R and R′ is independently selected from the group consisting of H, a linear alkyl group having 1-6 carbon atoms, a branched alkyl group having 2-12 carbon atoms, a cycloalkyl group having 3-17 carbon atoms, a fluorinated linear alkyl group having 2-12 carbon atoms, a fluorinated branched alkyl group having 2-12 carbon atoms, a fluorinated cycloalkyl group having 3-17 carbon atoms, an aryl group, an aralkyl group, an alkaryl group, a cycloalkyl group, a bicycloalkyl group, an alkenyl group, an alkalkenyl group, an alkenylalkyl group, an alkynyl group, an alkalkynyl group, an alkynylalkyl group, a trifluoropropyl group, a cyanopropyl group, an acryloyl group, an arylacryloyl group, an acryloylaryl group, an alkylacyl group, an arylacyl group, an alkylenylacyl group, and an alkynylacyl group, wherein any two R in the same molecule are optionally linked to form a three- to eight-membered cyclic group;

Z is a linear alkyl group having 1-6 carbon atoms;

each L is independently a linking group selected from the group consisting of linear or branched alkylene having 1 to 7 carbon atoms, cycloalkylene having 3 to 17 carbon atoms, alkylcycloalkylene having 4 to 20 carbon atoms, a cycloalkylalkylene having 4 to 20 carbon atoms, an arylene, having 4 to 30 carbon atoms, an alkylarylene, having 4 to 30 carbon atoms, an arylalkylene, having 4 to 30 carbon atoms, and combinations thereof;

each Ar is independently a bifunctional aromatic linking group wherein each Ar is selected from the group consisting of arylene, substituted arylene and heteroarylene;

n is 1, 2, or 3;

p is 0, 1, or 2; and

n+p≦3.

Hereinafter, Structures (VII), (VIII), (IX), (X), (XII), (XIII), (XIV), (XV), (XVI), (XVII), (XVIII), and (XIX) may represent all racemic forms and stereoisomers wherein said compounds may be capable of optical activity.

In one embodiment, each R in Structure (VII), (VIII), (IX), (X), (XII), (XIII), (XIV), (XV), (XVI), (XVII), (XVIII) or (XIX) is hydrogen.

In one embodiment, the nitrogen mustard is a nitrogen mustard of Structure (XVII). In another embodiment, the nitrogen mustard is a nitrogen mustard of Structure (XVII), wherein the Z in structure (XVII) is methyl or ethyl. In another embodiment, the nitrogen mustard is a nitrogen mustard of Structure (XVII), wherein each R in structure (XVII) is independently a linear alkyl group having 1-6 carbon atoms. In another embodiment, the nitrogen mustard is a nitrogen mustard of Structure (XVII), wherein the Z in structure (XVII) is methyl or ethyl and each R in structure (XVII) is independently a hydrogen or linear alkyl group having 1-6 carbon atoms.

In another embodiment, the nitrogen mustard of structure (XVII) is bis-(2-chloroethyl)ethylamine or bis-(2-chloroethyl)methylamine (also known as mechlorethamine).

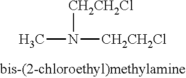

In one embodiment, the nitrogen mustard is a nitrogen mustard of Structure (IX). In another embodiment, the nitrogen mustard of Structure (IX) is tris-(2-chloroethyl)amine.

In one embodiment, the nitrogen mustard is a nitrogen mustard of Structure (XII). In another embodiment, the nitrogen mustard of structure (XII) is chlorambucil of Structure (XIIA):

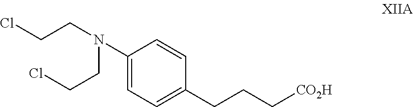 [SMILES: O=C(O)CCCc1ccc(N(CCCl)CCCl)cc1]

Structure (XII) may be cell cycle-phase nonspecific, although it also may be cytotoxic to nonproliferating cells. Activity may occur as a result of formation of an unstable ethylenimmonium ion, which alkylates or binds with many intracellular molecular structures, including nucleic acids. Its cytotoxic action may be primarily due to cross-linking of strands of DNA, which inhibits nucleic acid synthesis.

In one embodiment, the nitrogen mustard is a nitrogen mustard of Structure (XIII). In another embodiment, the nitrogen mustard of structure (XIII) is melphalan (also known as 4-bis(2-chloroethyl)amino-L-phenylalanine) of Structure (XIIIA):

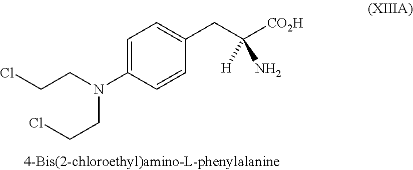 [SMILES: N[C@@H](Cc1ccc(N(CCCl)CCCl)cc1)C(=O)O]

Like the nitrogen mustards of Structure (XII), nitrogen mustards of Structure (XIII) may be cell cycle-phase nonspecific, although they also may be cytotoxic to nonproliferating cells.

In one embodiment, the nitrogen mustard is a nitrogen mustard of Structure (XVIII). In another embodiment, the nitrogen mustard of structure (XVIII) is uracil mustard of Structure (XVIIIA):

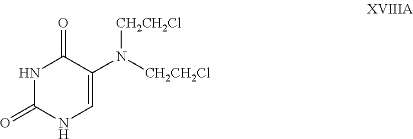

In another embodiment, the nitrogen mustard is in the form of a pharmaceutically acceptable salt. Suitable pharmaceutically acceptable salts of nitrogen mustard include HX salts of the following Structures (VIIa), (VIIIa), (IXa), (Xa), (XIIa), (XIIIa), (XIVa), (XVa), (XVIa), (XVIIa), (XVIIIa), and (XIXa):

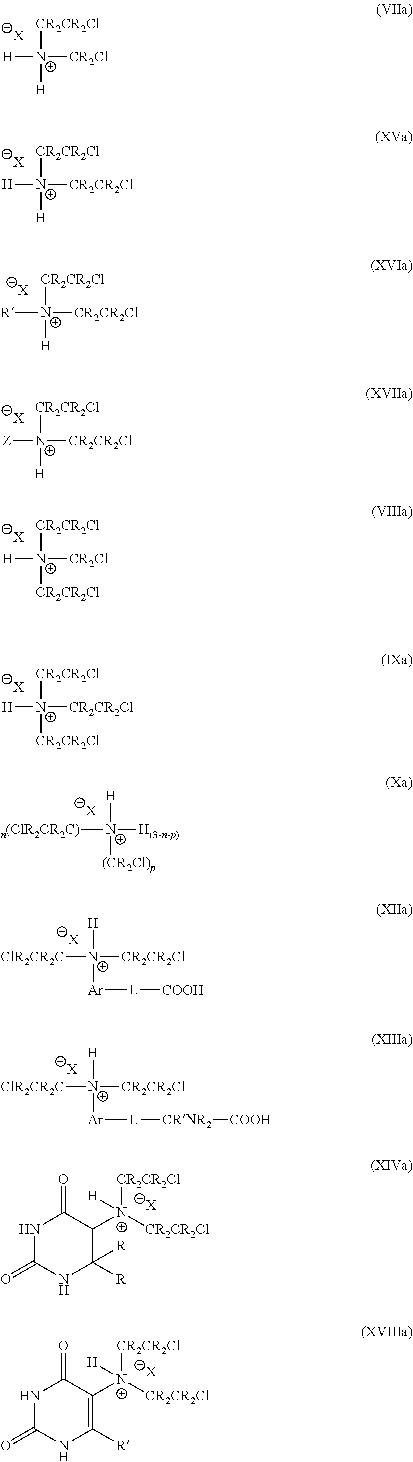

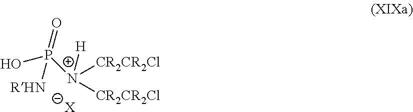

wherein R, R′. Z, Ar, L, n, and p are as defined above for the compounds of Structures (VII), (VIII), (IX), (X), (XII), (XIII), (XIV), (XV), (XVI), (XVIII), and (XIX).

In one embodiment, X⁻ is a halide, such as Cl⁻, Br⁻, or I⁻, or HSO₄⁻ or NO₃⁻. The corresponding HX is HCl, HBr, HI, or H₂SO₄, or HNO₃, respectively. In another embodiment, the pharmaceutically acceptable HX salt is a conventional acid-addition salt or base-addition salt formed from a non-toxic organic or inorganic acid or inorganic base. Illustrative acid-addition salts include those derived from inorganic acids such as hydrochloric acid, hydrobromic acid, hydroiodic acid, sulfuric acid, sulfamic acid, phosphoric acid, and nitric acid, and those derived from organic acids such as p-toluenesulfonic acid, methanesulfonic acid, ethane-disulfonic acid, isethionic acid, oxalic acid, p-bromophenylsulfonic acid, carbonic acid, succinic acid, citric acid, benzoic acid, 2-acetoxybenzoic acid, acetic acid, phenylacetic acid, propionic acid, glycolic acid, stearic acid, lactic acid, malic acid, tartaric acid, ascorbic acid, maleic acid, hydroxymaleic acid, glutamic acid, salicylic acid, sulfanilic acid, and fumaric acid. Illustrative base-addition salts include those derived from ammonium hydroxides (e.g., a quaternary ammonium hydroxide such as tetramethylammonium hydroxide), those derived from inorganic bases such as alkali or alkaline earth-metal (e.g., sodium, potassium, lithium, calcium, or magnesium) hydroxides, and those derived from non-toxic organic bases such as basic amino acids.

In another embodiment, the nitrogen mustard is provided in the form of a nitrogen mustard prodrug. Suitable nitrogen mustard prodrugs include those of the following Structure (XI):

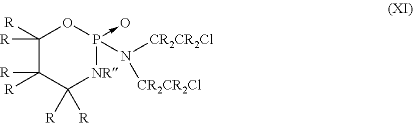

wherein each R and each R″ is independently selected from the group consisting of H, a linear alkyl group having 1-6 carbon atoms, a branched alkyl group having 2-12 carbon atoms, a cycloalkyl group having 3-17 carbon atoms, a fluorinated linear alkyl group having 2-12 carbon atoms, a fluorinated branched alkyl group having 2-12 carbon atoms, a fluorinated cycloalkyl group having 3-17 carbon atoms, an aryl group, an aralkyl group, an alkaryl group, a cycloalkyl group, a bicycloalkyl group, an alkenyl group, an alkalkenyl group, an alkenylalkyl group, an alkynyl group, an alkalkynyl group, an alkynylalkyl group, a trifluoropropyl group, a cyanopropyl group, an acryloyl group, an arylacryloyl group, an acryloylaryl group, an alkylacyl group, an arylacyl group, an alkylenylacyl group, and an alkynylacyl group, wherein any two R in the same molecule are optionally linked to form a three- to eight-membered cyclic group. Hereinafter, Structure (XI) may represent all racemic forms and stereoisomers wherein said compounds may be capable of optical activity.

For example, phosphatase and phosphamidase enzymes may cleave the P—N bond of Structure (XI), supra, e.g., cyclophosphamide, Structure (XIA), infra or ifosphamide, Structure (XIB), infra, resulting in an intermediate aldophosphamide, which may nonenzymatically break down to a bifunctional phosphoramide mustard, for example of Structure (XIXA) or (XIXB), as illustrated in Reactions 1a and 1b below. In an embodiment, cyclophosphamide, Structure (XIA), supra or ifosphamide, Structure (XIB), supra may be oxidatively activated by cytochrome P-450.

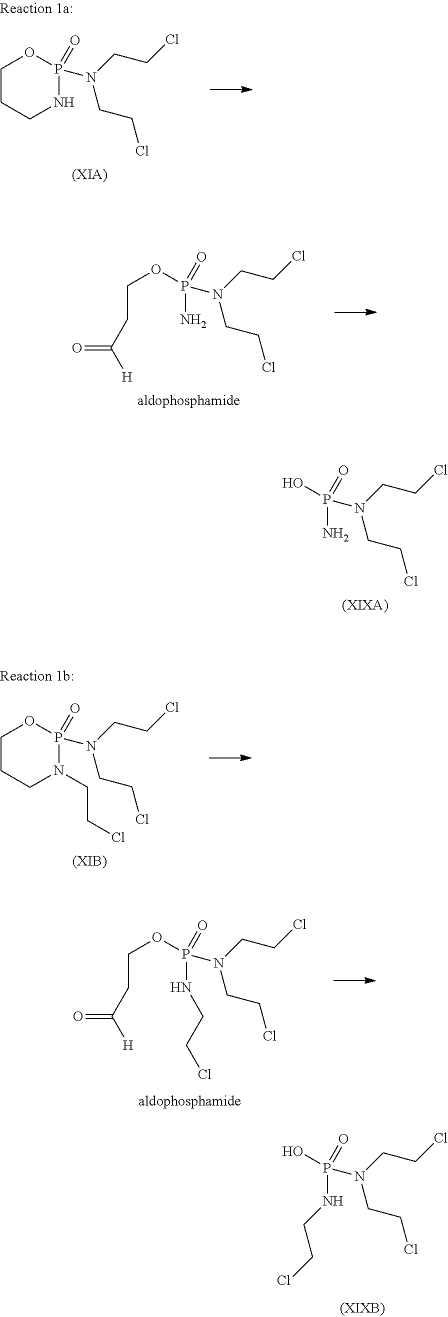

In one embodiment, the alkylating agent or pharmaceutically acceptable salt, solvate, or prodrug thereof is present in an amount of about 0.0001% to about 50%, about 0.0001% to about 25%, about 0.0001% to about 10%, or about 0.0001% to about 2% by weight of the composition. In another embodiment, the alkylating agent is present in an amount of about 0.001% to about 50%, about 0.001% to about 25%, about 0.001% to about 10%, or about 0.001% to about 2% by weight of the composition. In another embodiment, the alkylating agent is present in an amount of about 0.01% to about 10%, about 0.01% to about 2%, about 0.01% to about 1%, about 0.01% to about 0.08%, about 0.01% to about 0.06%, about 0.01 to about 0.5%, about 0.01% to about 0.04%, about 0.015% to about 0.04%, about 0.015% to about 0.03%, about 0.02%, or about 0.04% by weight of the composition.

B. Pharmaceutically Acceptable Excipients

Suitable pharmaceutically acceptable excipients include, but are not limited, to alcohols, ketones, aldehydes, ethers, amides, alkanes (linear, branched or cyclic), alkenes (linear, branched or cyclic), aromatics (fused or non-fused), dimethyl polysiloxanes, hydroxy ethers, substituted diols, ethylene glycol derivatives, polyoxylglycerides, polar aprotic solvents, alpha-hydroxycarboxylic acids and their salts, diesters of dibasic acids, polyethoxylated fatty acids, polyethylene glycol (“PEG”)-fatty acid diesters, PEG-fatty acid mono-ester and all-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterols and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, polyoxyethylenes, sorbitan fatty acid esters, lower alcohol fatty acid esters, ionic surfactants, penetration enhancers, and thickening agents.

Suitable alcohols include, but are not limited to, secondary alcohols and tertiary alcohols, such as isopropyl alcohol, cetyl alcohol, stearyl alcohol, cetearyl alcohol, and lanolin alcohol. Suitable alcohols also include ethanol, benzyl alcohol, 2,4-dichlorobenzyl alcohol, and alpha-terpinol, alpha-tocopherol, amerchol CAB, chlorobutanol (3,3,3,-trichloromethyl-2,2-dimethylethanol), hydroxyethyl cellulose, hydroxymethyl cellulose, hydroxyoctacosanyl hydroxystearate, hydroxypropyl cellulose (e.g., Krucel® hydroxypropyl cellulose), isostearyl alcohol, menthol, N,N-bis(2-hydroxyethyl)stearamide, octyl hydroxystearate, sorbitol, N-(2-Hydroxyethyl) pyrrolidone, or tetrahydrofurfuryl alcohol.

In one embodiment, the alcohol is a short chain aliphatic alcohol. In another embodiment, the short chain aliphatic alcohol is butyl alcohol, isopropyl alcohol, methyl alcohol, phenoxyethanol or tert butyl alcohol. In one embodiment, the alcohol is a long chain fatty alcohol. In another embodiment, the long chain fatty alcohol is ceteryl alcohol, cetyl alcohol, docosanol, myristyl alcohol, oleyl alcohol, or stearyl alcohol. In one embodiment, the alcohol is an amino alcohol. In another embodiment, the amino alcohol is 2-amino-2-methyl-1-propanol, diethanolamine, di-isopropanolamine, or monoethanolamine. In one embodiment, the alcohol is a C₁-C₂₅ alkanol, a C1-C12 alkanol, or a C1-C7 alkanol. In another embodiment, the alkanol has the formula: C₃-C₈ cycloalkyl-OH or C₃-C₈ heterocyclyl-OH, provided that the hydroxyl group is not attached to a carbon atom that is attached to a heteroatom.

Suitable ketones include, but are not limited to, acetone, ethyl methyl ketone and methyl isobutyl ketone.

Suitable dimethyl polysiloxanes include, but are not limited to, dimethicone and cyclodimethicone. In one embodiment, the dimethyl polysiloxane fluid has essentially no moisture content. As used herein, the term “dimethicone” includes low viscosity silicones, low viscosity, i.e. from about 1 cps to about 1,000 cps at 25° C. polydimethylsiloxanes, Hexamethyldisiloxane (CAS#107-46-0), pure silicone 1 cSt, volatile silicone, volatile silicones, volatile polydimethylsiloxanes, low temperature silicones, skin care silicone, skin care silicones, Octamethyltrisiloxane (CAS#107-51-7), Decamethyltetrasiloxane (CAS#141-62-8), Dodecamethylpentasiloxane (CAS#141-63-9), trisiloxane, low viscosity dimethicone, volatile dimethicone, cosmetic dimethicone fluid, cosmetic base fluids, suntan lotion silicone, antiperspirant silicone, hair care silicone, low surface tension silicone, and low heat of vaporization silicone. As used herein, the term “cyclomethicone” includes cyclopentasiloxane, volatile poydimethylcyclosiloxane (CAS#541-02-6), low surface tension silicone, volatile silicone, D5 silicone, Dow Corning 245 fluid, DC 245 fluid, 245 silicone, skin cream silicone, antiperspirant silicone, suntan lotion silicone, silicone for skin, skincare silicone, bodycare silicone, bath oil silicone, GE 1202, GE SF1202 cyclopentasiloxane, D5 Cyclopentasiloxane, and D5 Decamethylcyclopentasiloxane.

In one embodiment, the pharmaceutically acceptable excipient is a substituted diol. In one embodiment, the substituted diol is a compound of the formula

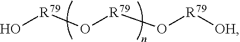

wherein n is 1 to 6, and each R⁷⁹ is independently selected from the group consisting of a linear alkylene group having 1-12 carbon atoms, a branched alkylene group having 3-12 carbon atoms, a linear gem-disubstituted alkylene group having 1-12 carbon atoms, a branched gem-disubstituted alkylene group having 3-12 carbon atoms, a cyclic gem-disubstituted alkylene group having 3-12 carbon atoms, a cycloalkylene group having 3-12 carbon atoms, a fluorinated linear alkylene group having 2-12 carbon atoms, a fluorinated branched alkylene group having 3-12 carbon atoms, and a fluorinated cycloalkylene group having 3-12 carbon atoms, an arylene group, an aralkylene group, an alkarylene group, a cycloalkylene group, a bicycloalkylene group, an alkenylene group, an alkalkenylene group, an alkenylalkylene group, an alkynylene group, an alkynylalkylene group, a trifluoropropylene group, a cyanopropylene group, an acryloyl group, an arylacryloyl group, an acryloylarylene group, a carboalkylene group, carboarylene group, a carboalkenylene group and a carboalkynylene group, and combinations thereof. Each —OH group of the diol is independently primary, secondary, or tertiary. In some embodiments, all R⁷⁹ groups within the same molecule are identical. In some embodiments, the R⁷⁹ groups within the same molecule can be the same or different.

In one embodiment, n is 1 and the substituted diol is a compound of the formula

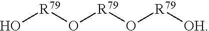

In another embodiment, each R⁷⁹ is independently a linear alkylene group having 1-12 carbon atoms, or a branched alkylene group having 3-12 carbon atoms. In another embodiment, one or more R⁷⁹ is independently a linear alkylene group having 1-12 carbon atoms. In another embodiment, one or more R⁷⁹ is independently a linear alkylene group having 1-6 carbon atoms. In another embodiment, one or more R⁷⁹ is independently a linear alkylene group having 2 to 3 carbon atoms. In another embodiment, one or more R⁷⁹ is independently a linear alkylene group having 4 to 5 carbon atoms. In another embodiment, each R⁷⁹ is ethylene.

In another embodiment, one or more R⁷⁹ is independently a branched alkylene group having 3-12 carbon atoms. In another embodiment, one or more R⁷⁹ is independently a branched alkylene group having 3-6 carbon atoms. In another embodiment, one or more R⁷⁹ is independently a branched alkylene group having 3 to 4 carbon atoms. In another embodiment, one or more R⁷⁹ is independently a branched alkylene group having 4 to 5 carbon atoms.

In another embodiment, the diol has one of the following structures:

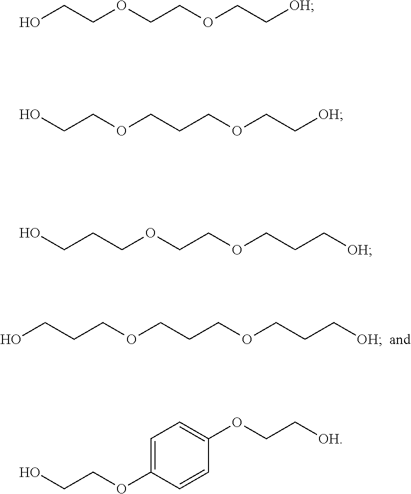

In some embodiments, each R⁷⁹ is independently a group of the formula:

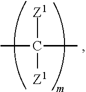

wherein each Z¹ is independently H, linear C₁-C₁₂ alkyl, branched C₃-C₁₂ alkyl, cyclic C₃-C₁₂ alkyl, linear C₂-C₁₂ alkenyl, branched C₃-C₁₂ alkenyl, cyclic C₅-C₁₂ alkenyl, linear C₂-C₁₂ alkynyl, branched C₄-C₁₂ alkynyl, cyclic C₈-C₁₂ alkynyl, aryl, heteroaryl, or heterocyclyl, any of which is optionally-substituted with any number of halogens, or two vicinal or geminal Z¹ groups can together form a carbocyclic or heterocyclic ring with the carbon atom(s) to which the Z¹ groups are attached; and m is 1-12. In some embodiments, m is 1, 2, 3, 4, 5, 6, 7, 8, 9, 10, 11, or 12. In some embodiments, m is 2.

In some embodiments, the diol is a compound of the formula:

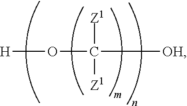

wherein each Z¹ is independently H, linear C₁-C₁₂ alkyl, branched C₃-C₁₂ alkyl, cyclic C₃-C₁₂ alkyl, linear C₂-C₁₂ alkenyl, branched C₃-C₁₂ alkenyl, cyclic C₅-C₁₂ alkenyl, linear C₂-C₁₂ alkynyl, branched C₄-C₁₂ alkynyl, cyclic C₈-C₁₂ alkynyl, aryl, aralkyl, alkaryl, heteroaryl, hetereoarylalkyl, alkheteroaryl, heterocyclyl, heterocyclylalkyl, or alkheterocyclyl, any of which is optionally-substituted with any number of halogens, or two vicinal or geminal Z¹ groups can together form a carbocyclic or heterocyclic ring with the carbon atom(s) to which the Z¹ groups are attached; each m is independently 1-12; and n is 3-8. In some embodiments, each m is 1, 2, 3, 4, 5, 6, 7, 8, 9, 10, 11, or 12. In some embodiments, each m is 2. In some embodiments, n is 3, 4, 5, 6, 7, or 8. In some embodiments, n is 3. The formula provides for: i) termini which are independently primary, secondary, or tertiary hydroxyl groups; and ii) subunits, each of which is independently derived from a primary/primary diol, a secondary/secondary diol, a tertiary/tertiary diol, a primary/secondary diol, a primary/tertiary diol, or a secondary/tertiary diol.

Non-limiting examples of Z¹ groups include H, methyl, ethyl, 2,2,2-trifluoroethyl, 2,2,2-trichloroethyl, propyl, butyl, isopropyl, isobutyl, sec-butyl, pentyl, hexyl, ethenyl, acetylenyl, cyclopropyl, cyclobutyl, cyclopentyl, cyclohexyl, phenyl, benzyl, pyridyl, picolyl, furanyl, tetrahydrofuranyl, imidazolyl), thiophenyl, oxazolyl, isoxazolyl, and pyrrolyl.

In some embodiments, the diol is a compound of the formula:

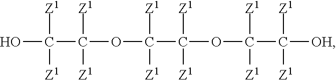

wherein each Z¹ is independently H, linear C₁-C₁₂ alkyl, branched C₃-C₁₂ alkyl, cyclic C₃-C₁₂ alkyl, linear C₂-C₁₂ alkenyl, branched C₃-C₁₂ alkenyl, cyclic C₅-C₁₂ alkenyl, linear C₂-C₁₂ alkynyl, branched C₄-C₁₂ alkynyl, cyclic C₈-C₁₂ alkynyl, aryl, aralkyl, alkaryl, heteroaryl, hetereoarylalkyl, alkheteroaryl, heterocyclyl, heterocyclylalkyl, or alkheterocyclyl, any of which is optionally-substituted with any number of halogens, or two vicinal or geminal Z¹ groups can together form a carbocyclic or heterocyclic ring with the carbon atom(s) to which the Z¹ groups are attached. Based on the formula, one of skill in the art can envision: i) termini which are independently primary, secondary, or tertiary hydroxyl groups; and ii) subunits, each of which is independently derived from a primary/primary diol, a secondary/secondary diol, a tertiary/tertiary diol, a primary/secondary diol, a primary/tertiary diol, or a secondary/tertiary diol.

Non-limiting examples of diols include:

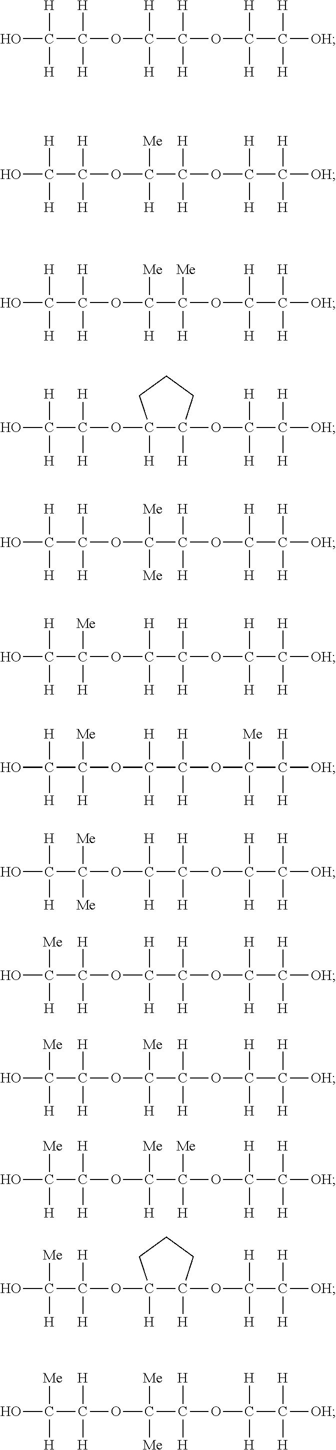

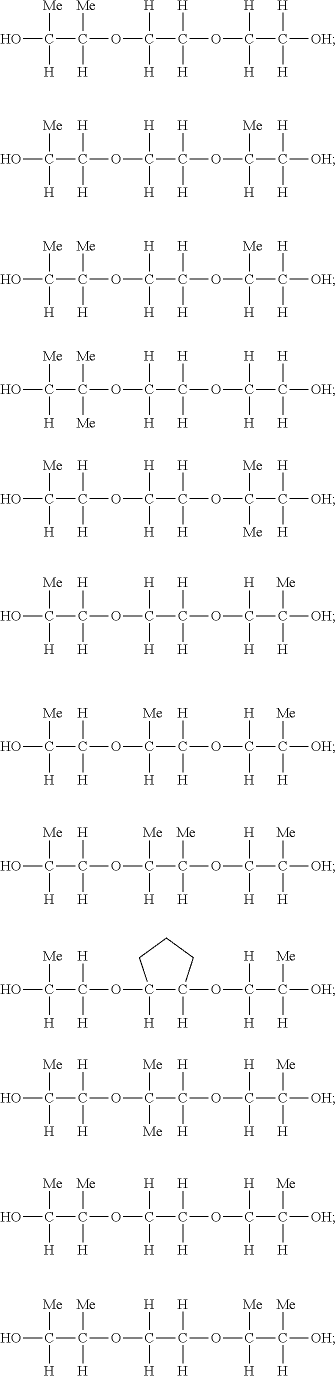

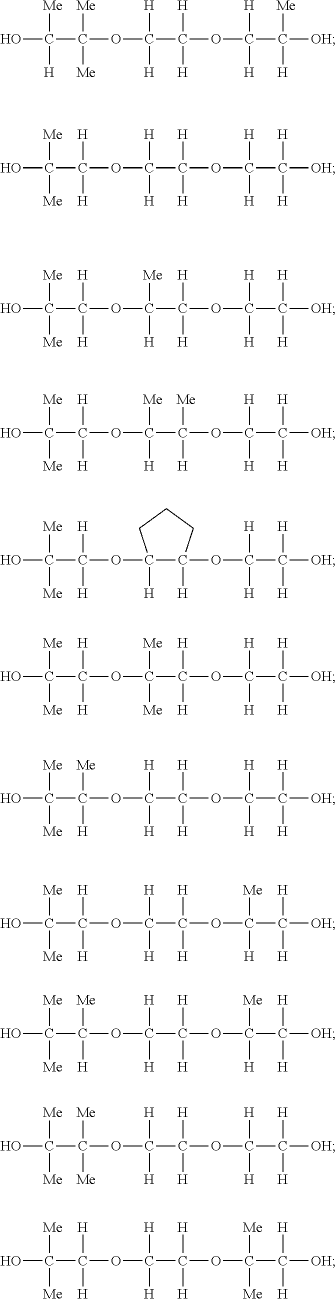

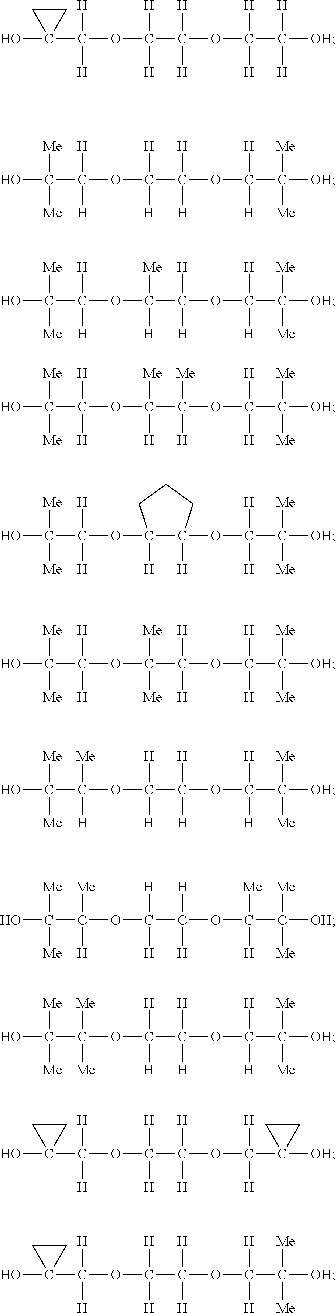

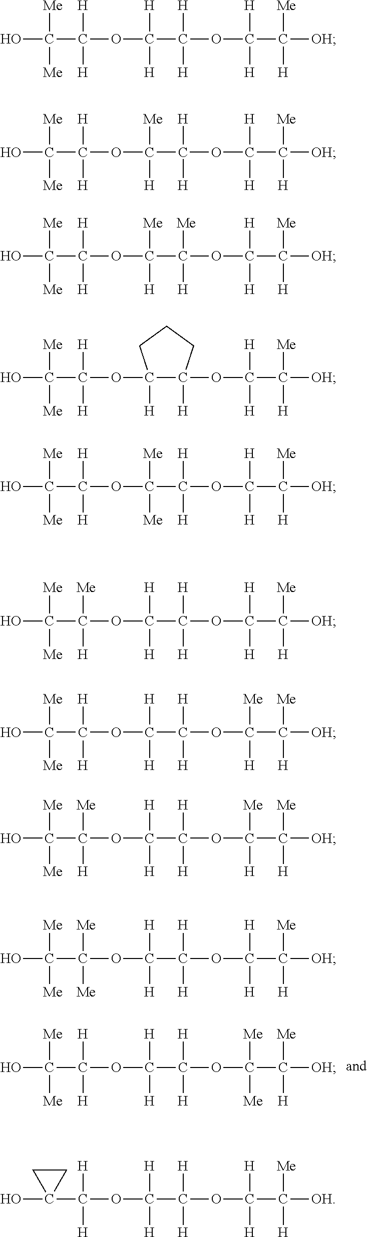

The structures contemplate all possible stereochemistries, stereoisomers, and mixtures thereof.

In one embodiment, the pharmaceutically acceptable excipient is a hydroxy ether. In one embodiment, the hydroxy ether is a compound of the formula

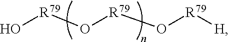

wherein R⁷⁹ is as defined above and n is an integer from 1 to 6. The —OH group of the hydroxy ether is a primary, secondary, or tertiary hydroxyl group. In some embodiments, all R⁷⁹ groups within the same molecule are identical. In some embodiments, the R⁷⁹ groups within the same molecule can be the same or different.

In one embodiment, n is 1, and the hydroxy ether is a compound of the formula

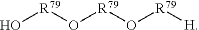

In another embodiment, each R⁷⁹ is independently a linear alkylene group having 1-12 carbon atoms, or a branched alkylene group having 3-12 carbon atoms. In another embodiment, one or more R⁷⁹ is independently a linear alkylene group having 1-12 carbon atoms. In another embodiment, one or more R⁷⁹ is independently a linear alkylene group having 1-6 carbon atoms. In another embodiment, one or more R⁷⁹ is independently a linear alkylene group having 2 to 3 carbon atoms. In another embodiment, one or more R⁷⁹ is independently a linear alkylene group having 4 to 5 carbon atoms. In another embodiment, each R⁷⁹ is ethylene.

In another embodiment, one or more R⁷⁹ is independently a branched alkylene group having 3-12 carbon atoms. In another embodiment, one or more R⁷⁹ is independently a branched alkylene group having 3-6 carbon atoms. In another embodiment, one or more R⁷⁹ is independently a branched alkylene group having 3 to 4 carbon atoms. In another embodiment, one or more R⁷⁹ is independently a branched alkylene group having 4 to 5 carbon atoms. In another embodiment, each R⁷⁹ is ethylene and the hydroxy ether has the following structure:

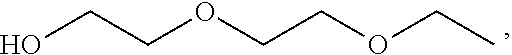

which is 2-(2-ethoxyethoxy)ethanol (also known as diethylene glycol monoethyl ether, 2-(2-ethoxyethoxy)ethanol or Transcutol®). Other non-limiting examples of suitable hydroxy ethers include:

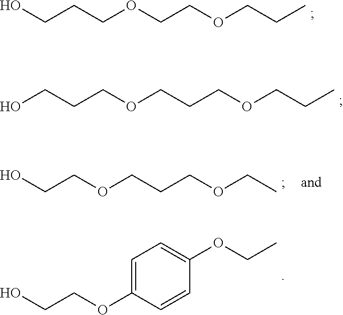

In some embodiments, each R⁷⁹ is independently a group of the formula:

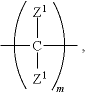

wherein each Z¹ is independently H, linear C₁-C₁₂ alkyl, branched C₃-C₁₂ alkyl, cyclic C₃-C₁₂ alkyl, linear C₂-C₁₂ alkenyl, branched C₃-C₁₂ alkenyl, cyclic C₅-C₁₂ alkenyl, linear C₂-C₁₂ alkynyl, branched C₄-C₁₂ alkynyl, cyclic C₈-C₁₂ alkynyl, aryl, heteroaryl, or heterocyclyl, any of which is optionally-substituted with any number of halogens, or two vicinal or geminal Z¹ groups can together form a carbocyclic or heterocyclic ring with the carbon atom(s) to which the Z¹ groups are attached; and m 1-2. In some embodiments, m is 1, 2, 3, 4, 5, 6, 7, 8, 9, 10, 11, or 12. In some embodiments, m is 2.

In some embodiments, the hydroxy ether is a compound of the formula:

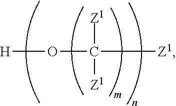

wherein each Z¹ is independently H, linear C₁-C₁₂ alkyl, branched C₃-C₁₂ alkyl, cyclic C₃-C₁₂ alkyl, linear C₂-C₁₂ alkenyl, branched C₃-C₁₂ alkenyl, cyclic C₅-C₁₂ alkenyl, linear C₂-C₁₂ alkynyl, branched C₄-C₁₂ alkynyl, cyclic C₈-C₁₂ alkynyl, aryl, aralkyl, alkaryl, heteroaryl, hetereoarylalkyl, alkheteroaryl, heterocyclyl, heterocyclylalkyl, or alkheterocyclyl, any of which is optionally-substituted with any number of halogens, or two vicinal or geminal Z¹ groups can together form a carbocyclic or heterocyclic ring with the carbon atom(s) to which the Z¹ groups are attached; each m is independently 1-12, and n is 3-8. In some embodiments, each m is 1, 2, 3, 4, 5, 6, 7, 8, 9, 10, 11, or 12. In some embodiments, each m is 2. In some embodiments, n is 3, 4, 5, 6, 7, or 8. In some embodiments, n is 3. The formula provides for i) a terminal hydroxyl group, which is primary, secondary, or tertiary; ii) subunits, each of which is independently derived from a primary/primary diol, a secondary/secondary diol, a tertiary/tertiary diol, a primary/secondary diol, a primary/tertiary diol, or a secondary/tertiary diol; and iii) a non-hydroxyl terminus derived from a primary, secondary, or tertiary alcohol.

Non-limiting examples of Z¹ groups include H, methyl, ethyl, 2,2,2-trifluoroethyl, 2,2,2-trichloroethyl, propyl, butyl, isopropyl, isobutyl, sec-butyl, pentyl, hexyl, ethenyl, acetylenyl, cyclopropyl, cyclobutyl, cyclopentyl, cyclohexyl, phenyl, benzyl, pyridyl, picolyl, furanyl, tetrahydrofuranyl, imidazolyl), thiophenyl, oxazolyl, isoxazolyl, and pyrrolyl.

In some embodiments, the hydroxy ether is a compound of the formula:

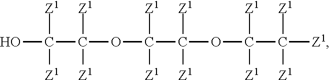

wherein each Z¹ is independently H, linear C₁-C₁₂ alkyl, branched C₃-C₁₂ alkyl, cyclic C₃-C₁₂ alkyl, linear C₂-C₁₂ alkenyl, branched C₃-C₁₂ alkenyl, cyclic C₅-C₁₂ alkenyl, linear C₂-C₁₂ alkynyl, branched C₄-C₁₂ alkynyl, cyclic C₈-C₁₂ alkynyl, aryl, aralkyl, alkaryl, heteroaryl, hetereoarylalkyl, alkheteroaryl, heterocyclyl, heterocyclylalkyl, or alkheterocyclyl, any of which is optionally-substituted with any number of halogens. Based on the formula, one of skill in the art can envision: i) a terminal hydroxyl group, which is primary, secondary, or tertiary; ii) subunits, each of which is independently derived from a primary/primary diol, a secondary/secondary diol, a tertiary/tertiary diol, a primary/secondary diol, a primary/tertiary diol, or a secondary/tertiary diol; and iii) a non-hydroxyl terminus derived from a primary, secondary, or tertiary alcohol.

Non-limiting examples of hydroxy ethers include:

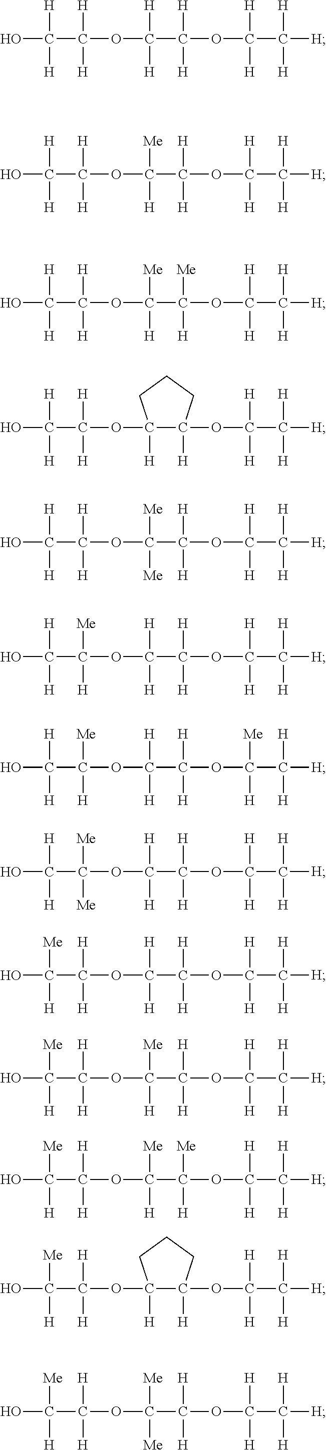

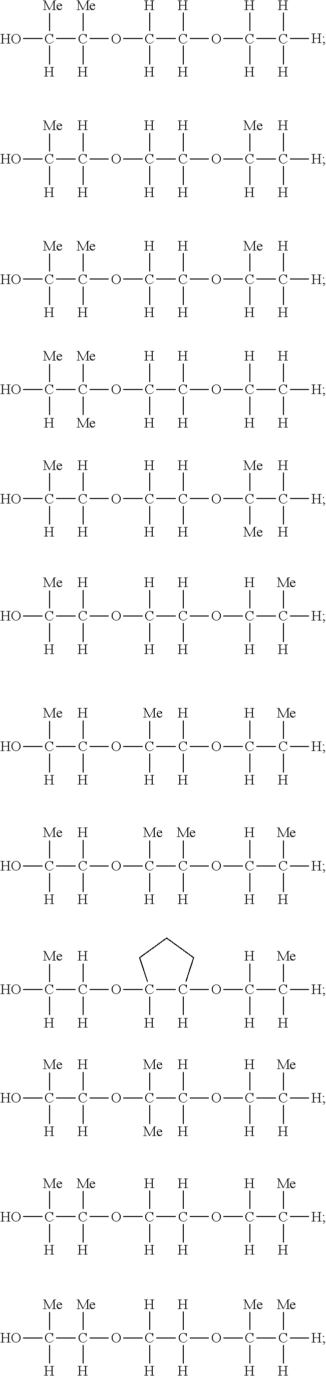

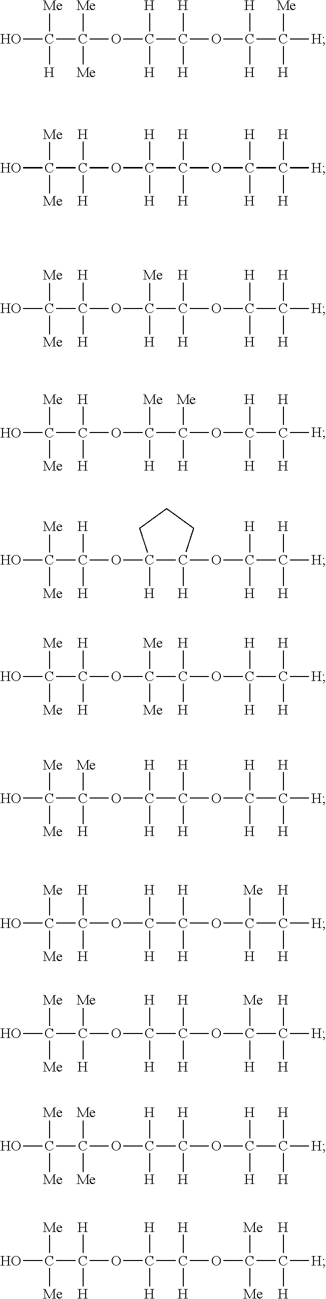

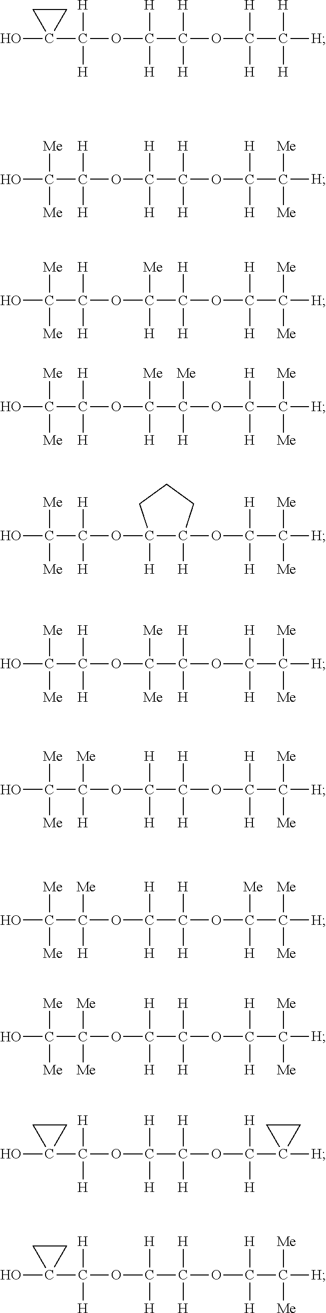

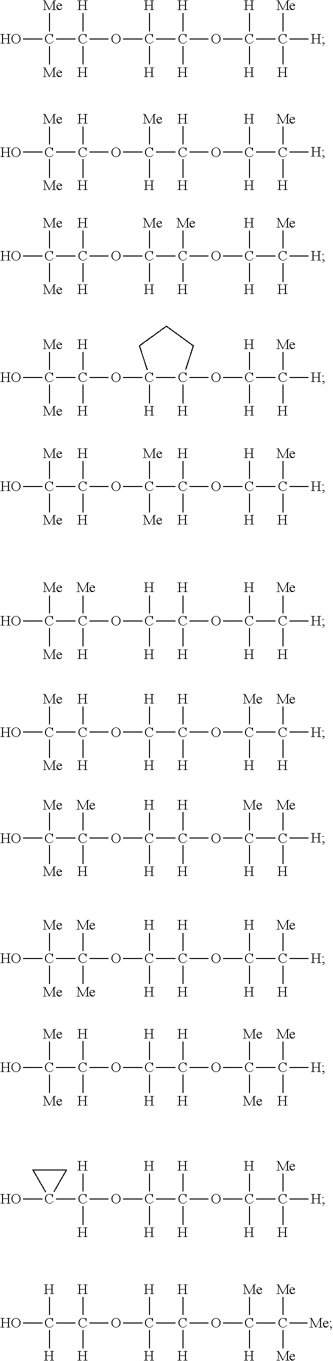

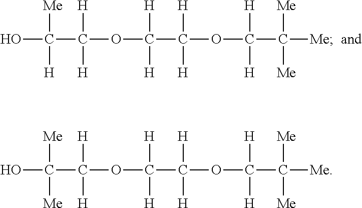

In another embodiment, the ethylene glycol derivative is a diol or a diol derivative that is butylene glycol, dipropylene glycol, ethylene glycol, hexylene glycol, propylene glycol, ethyl hexanediol, propylene glycol monolaurate, propylene glycol monostearate, propylene glycol palmitostearate or propylene glycol ricinoleate. In one embodiment, the ethylene glycol derivative is a polyol that is glycerin, glyceryl acetate, glyceryl citrate, glyceryl isostearate, glyceryl laurate, glyceryl monostearate, glyceryl oleate, glyceryl palmitate, glyceryl ricinoleate, glyceryl stearate-laureth-23, glyceryl stearate SE, glyceryl stearate/PEG-100 stearate or 1,2,6-hexanetriol.

In another embodiment, the ethylene glycol derivative has the formula R-alkylene-R or R— heteroalkylene-R wherein R is a hydroxy, alkoxy, phenoxy, alkylcarbonyloxy or arylcarbonyloxy, and wherein the alkylene or heteroalkylene moiety is optionally substituted with a hydroxyl, alkoxy, phenoxy, alkylcarbonyloxy or arylcarbonyloxy, provided however that, 2 heteroatoms (for example, 2 oxygen atoms) are not attached to the same carbon atom. The alkylene moiety can be C₁-C₇ alkylene, C₁-C₅ alkylene, or C₁-C₃ alkylene. The heteroalkylene moiety can be C₁-C₇ heteroalkylene, C₁-C₅ heteroalkylene or C₁-C₃ heteroalkylene.

In another embodiment, the ethylene glycol derivative is a polyethylene glycol (PEG) moiety that is polyethylene glycol 1000, polyethylene glycol 1500, polyethylene glycol 1540, polyethylene glycol 200, polyethylene glycol 300, polyethylene glycol 300-1600, polyethylene glycol 3350, polyethylene glycol 400, polyethylene glycol 4000, polyethylene glycol 540, polyethylene glycol 600, polyethylene glycol 6000, polyethylene glycol 8000, polyethylene glycol 900, polyoxyethylene-polyoxypropylene 1800 or a polyoxyethylene alcohol.

In another embodiment, the ethylene glycol derivative is a PEG derivative that is ceteth-2, ceteth-10, ceteth-20, ceteth-23, dimethicone copolyol, PEG 6-32 stearate/glycol stearate, PEG-22 methyl ether/dodecyl glycol copolymer, PEG-25 propylene glycol stearate, PEG-45/dodecyl glycol copolymer, peglicol-5-oleate, pegoxol 7 stearate, PPG-12/SMDI copolymer, polypropylene glycol (PPG)-15 stearyl ether, PPG-20 methyl glucose ether distearate, PPG-26 oleate, steareth-10, steareth-100, steareth-2, steareth-20, or steareth-21. In another embodiment, the ethylene glycol derivative is a PEG derivative that is poloxamer 124, poloxamer 181, poloxamer 182, poloxamer 188, poloxamer 237, poloxamer 407, a polyoxyethylene fatty acid ester, polyoxyethylene propylene, methoxypolyoxyethylene glycol 350 or tyloxapol. In another embodiment, the ethylene glycol derivative is a PEG derivative that is nonoxynol-15, nonoxynol-15, nonoxynol-9, octoxynol-1 or octoxynol-9. In another embodiment, the ethylene glycol derivative is a PEG derivative that is polyoxyl 100 glyceryl stearate, polyoxyl 100 stearate, polyoxyl 15 cocamine, polyoxyl 150 distearate, polyoxyl 2 stearate, polyoxyl 4 dilaurate, polyoxyl 40 hydrogenated castor oil, polyoxyl 40 stearate, polyoxyl 400 stearate, polyoxyl 50 stearate, polyoxyl 6 and polyoxyl 32 palmitostearate, polyoxyl 6 isostearate, polyoxyl 60 hydrogenated castor oil, polyoxyl 75 lanolin, polyoxyl 8 laurate, polyoxyl 8 stearate, polyoxyl distearate, polyoxyl glyceryl stearate, polyoxyl lanolin or polyoxyl stearate.

Suitable polyoxylglycerides include, but are not limited to, caprylocaproyl, linoleoyl, oleoyl, lauroyl, and stearoyl polyoxylglycerides. In one embodiment, the polyoxylglyceride is lauroyl polyoxyl-32 glycerides, stearoyl polyoxyl-32 glycerides, medium chain triglycerides, oleoyl polyoxyl-6 glycerides, linoleoyl polyoxyl-6 glycerides, lauroyl polyoxyl-6 glycerides, or caprylocaproyl polyoxyl-8-glycerides. Such polyoxylglycerides are available from Gattefosse (Canada) under the tradenames Labrasol®, Labrafil®, and Gelucire®.

Suitable polar aprotic solvents include, but are not limited to dimethyl sulfoxide (DMSO) and propylene carbonate (PC), ethylene carbonate, glycerin carbonate, N-methyl pyrrolidone, and dimethyl acetamide.

Suitable alpha-hydroxycarboxylic acids include alpha-hydroxycarboxylic acid having from 2 to 25, from 5 to 20, from 10 to 15, and from 2 to 5 carbon atoms. The alkylene backbone of such acids can be suitably substituted. Suitable substituents include, without limitation, alkoxy, amino, halo, hydroxy and phenoxy groups. In one embodiment, the alpha-hydroxycarboxylic acid is lactic acid, glycolic acid or malic acid. Salts of the alpha-hydroxycarboxylic acids include, without limitation, sodium salt and potassium salt; alkaline earth metal salts, such as calcium salt and magnesium salt; amine salts, such as ammonium salt, triethylamine salt, and triethanol amine salt; and basic amino acid salts, such as arginine salt and lysine salt.

Suitable diesters of dibasic acids typically comprise as part of the ester moiety, alcohols having from 1 to 25, from 5 to 20, from 10 to 15, and from 1 to 4 carbon atoms. In one embodiment, the alcohol useful as part of the diester of a dibasic acid is methyl alcohol, ethyl alcohol, propyl alcohol, isopropyl alcohol, butyl alcohol, isobutyl alcohol or tert-butyl alcohol. In another embodiment, the dibasic acids are those having from 1 to 25, from 5 to 20, from 10 to 15, or from 1 to 10 carbon atoms. In another embodiment, the dibasic acid includes, but is not limited to, adipic acid, sebacic acid, oxalic acid, carbonic acid, diethyl adipate, di-isopropyl adipate, diethyl sebacate, or di-isopropyl sebacate.

Suitable polyethoxylated fatty acids include, but are not limited to, PEG 4-100 monolaurate (Crodet L series, Croda), PEG 4-100 monooleate (Crodet 0 series, Croda), PEG 4-100 monostearate (Crodet S series, Croda, and Myrj Series, Atlas/ICI), PEG 400 distearate (Cithrol 4DS series, Croda), PEG 100, 200, or 300 monolaurate (Cithrol ML series, Croda), PEG 100, 200, or 300 monooleate (Cithrol MO series, Croda), PEG 400 dioleate (Cithrol 4DO series, Croda), PEG 400-1000 monostearate (Cithrol MS series, Croda), PEG-1 stearate (Nikkol MYS-IEX, Nikko, and Coster Kl, Condea), PEG-2 stearate (Niklol MYS-2, Nikko), PEG-2 oleate (Nikkol MYO-2, Nikko), PEG-4 laurate (Mapeg( ) 200 ML, PPG), PEG-4 oleate (Mapeg( ) 200 MO, PPG), PEG-4 stearate (Kessco PEG 200 MS, Stepan), PEG-5 stearate (Nikkol TMGS-5, Nikko), PEG-5 oleate (Nikkol TMGO-5, Nikko), PEG-6 oleate (Argon OL 60, Auschem SpA), PEG-7 oleate (Argon OL 70, Auschem SpA), PEG-6 laurate (Kessco PEG300 ML, Stepan), PEG-7 laurate (Lauridac 7, Condea), PEG-6 stearate (Kessco, PEG300 MS, Stepan), PEG-8 laurate (Mapeg( ) 400 ML, PPG), PEG-8 oleate (Mapeg 400 MO, PPG), PEG-8 stearate (Mapeg) 400 MS, PPG), PEG-9 oleate (Emulgante A9, Condea), PEG-9 stearate (Cremophor S9, BASF), PEG-10 laurate (Nikkol MYL-10, Nikko), PEG-10 oleate (Nikkol MYO-10, Nikko), PEG-12 stearate (Nikkol MYS-10, Nikko), PEG-12 laurate (Kessco PEG 600 ML, Stepan), PEG-12 oleate (Kessco PEG 600 MO, Stepan), PEG-12 ricinoleate (CAS 9004-97-1), PEG-12 stearate (Mapeg 600 MS, PPG), PEG-15 stearate (Nikkol TMGS-15, Nikko), PEG-15 oleate (Nikkol TMGO-15, Nikko), PEG-20 laurate (Kessco PEG 1000 ML, Stepan), PEG-20 oleate (Kessco PEG 1000 MO, Stepan), PEG-20 stearate (Mapeg 1000 MS, PPG), PEG-25 stearate (Nikkol MYS-25, Nikko), PEG-32 laurate (Kessco@D PEG 1540 ML, Stepan), PEG-32 oleate (Kessco) PEG 1540 MO, Stepan), PEG-32 stearate (Kessco PEG 1540 MS, Stepan), PEG-30 stearate (Myrj 51), PEG-40 laurate (Crodet L40, Croda), PEG-40 oleate (Crodet 040, Croda), PEG-40 stearate (Emerest 2715, Henkel), PEG-45 stearate (Nikkol MYS-45, Nikko), PEG-50 stearate (Myrj 53), PEG-55 stearate (Nikkol MYS 55, Nikko), PEG-100 oleate (Crodet 0-100, Croda), PEG-100 stearate (Ariacel 165, ICI), PEG-200 oleate (Albunol 200 MO, Taiwan Surf.), PEG-400 oleate (LACTOMUL, Henkel), and PEG-600 oleate (Albunol 600 MO, Taiwan Surf.).

Suitable PEG-fatty acid diesters include, but are not limited to, PEG-4 dilaurate (Mapeg) 200 DL, PPG), PEG-4 dioleate (Mapeg 200 DO, PPG), PEG-4 distearate (Kessco 200 DS, Stepan), PEG-6 dilaurate (Kessco PEG 300 DL, Stepan), PEG-6 dioleate (Kessco PEG 300 DO, Stepan), PEG-6 distearate (Kessco PEG 300 DS, Stepan), PEG-8 dilaurate (Mapeg 400 DL, PPG), PEG-8 dioleate (Ma peg 400 DO, PPG), PEG-8 distearate (Ma peg 400 DS, PPG), PEG-10 dipalmitate (Polyaldo 2PKFG), PEG-12 dilaurate (Kessco PEG 600 DL, Stepan), PEG-12 distearate (Kessco PEG 600 DS, Stepan), PEG-12 dioleate (Mapeg) 600 DO, PPG), PEG-20 dilaurate (Kessco PEG 1000 DL, Stepan), PEG-20 dioleate (Kessco) PEG 1000 DO, Stepan), PEG-20 distearate (Kessco PEG 1000 DS, Stepan), PEG-32 dilaurate (Kessco PEG 1540 DL, Stepan), PEG-32 dioleate (Kessco) PEG 1540 DO, Stepan), PEG-32 distearate (Kessco PEG 1540 DS, Stepan), PEG-400 dioleate (Cithrol 4DO series, Croda), and PEG-400 distearate Cithrol 4DS series, Croda).

Suitable PEG-fatty acid mono- and all-ester mixtures include, but are not limited to, PEG 4-150 mono, dilaurate (Kessco PEG 200-6000 mono, Dilaurate, Stepan), PEG 4-150 mono, dioleate (Kessco PEG 200-6000 mono, Dioleate, Stepan), and PEG 4-150 mono, distearate (Kessco 200-6000 mono, Distearate, Stepan).

Suitable polyethylene glycol glycerol fatty acid esters include, but are not limited to, PEG-20 glyceryl laurate (Tagat) L, Goldschmidt), PEG-30 glyceryl laurate (Tagat L2, Goldschmidt), PEG-15 glyceryl laurate (Glycerox L series, Croda), PEG-40 glyceryl laurate (Glycerox L series, Croda), PEG-20 glyceryl stearate (Capmul EMG, ABITEC), and Aldo MS-20 KFG, Lonza), PEG-20 glyceryl oleate (Tagat O, Goldschmidt), and PEG-30 glyceryl oleate (Tagat 02, Goldschmidt).

Suitable alcohol-oil transesterification products include, but are not limited to, PEG-3 castor oil (Nikkol CO-3, Nikko), PEG-5, 9, and 16 castor oil (ACCONON CA series, ABITEC), PEG-20 castor oil, (Emalex C-20, Nihon Emulsion), PEG-23 castor oil (Emulgante EL23), PEG-30 castor oil (Incrocas 30, Croda), PEG-35 castor oil (Incrocas-35, Croda), PEG-38 castor oil (Emulgante EL 65, Condea), PEG-40 castor oil (Emalex C-40, Nihon Emulsion), PEG-50 castor oil (Emalex C-50, Nihon Emulsion), PEG-56 castor oil (Eumulgin PRT 56, Pulcra SA), PEG-60 castor oil (Nikkol CO-60TX, Nikko), PEG-100 castor oil, PEG-200 castor oil (Eumulgin PRT 200, Fulcra SA), PEG-5 hydrogenated castor oil (Nikkol HCO-5, Nikko), PEG-7 hydrogenated castor oil (Cremophor W07, BASF), PEG-10 hydrogenated castor oil (Nikkol HCO-IO, Nikko), PEG-20 hydrogenated castor oil (Nikkol HCO-20, Nikko), PEG-25 hydrogenated castor oil (Simulsol 1292, Seppic), PEG-30 hydrogenated castor oil (Nikkol HCO 30, Nikko), PEG-40 hydrogenated castor oil (Cremophor RH 40, BASF), PEG hydrogenated castor oil (Cerex ELS 450, Auschem Spa), PEG-50 hydrogenated castor oil (Emalex HC-50, Nihon Emulsion), PEG-60 hydrogenated castor oil (Nikkol HCO-60, Nikko), PEG-80 hydrogenated castor oil (Nikkol HCO-80, Nikko), PEG-100 hydrogenated castor oil (Nikkol HCO 100, Nikko), PEG-6 corn oil (Labrafl M 2125 CS, Gattefosse), PEG-6 almond oil (Labrafil M 1966 CS, Gattefosse), PEG-6 apricot kernel oil (Labrafl M 1944 CS, Gattefosse), PEG-6 olive oil (Labrafil) M 1980 CS, Gattefosse), PEG-6 peanut oil (Labrafil) M 1969 CS, Gattefosse), PEG-6 hydrogenated palm kernel oil (Labrafil M 2130 BS, Gattefosse), PEG-6 palm kernel oil (Labrafil M 2130 CS, Gattefosse), PEG-6 triolein (Labrafl M 2735 CS, Gattefosse), PEG-8 corn oil (Labrafl WL 2609 BS, Gattefosse), PEG-20 corn glycerides (Crovol M40, Croda), PEG-20 almond glycerides (Crovol A40, Croda), PEG-25 trioleate (TAGAT TO, Goldschmidt), PEG-40 palm kernel oil (Crovol PK-70), PEG-60 corn glycerides (Crovol M70, Croda), PEG-60 almond glycerides (Crovol A70, Croda), PEG-4 caprylic/capric triglyceride (Labrafac( ) Hydro, Gattefosse), PEG-8 caprylic/capric glycerides (Labrasol, Gattefosse), PEG-6 caprylic/capric glycerides (SOFTIGEN767, HuIs), lauroyl macrogol-32 glyceride (GELUCIRE 44/14, Gattefosse), stearoyl macrogol glyceride (GELUCIRE 50/13, Gattefosse), mono, di, tri, tetra esters of vegetable oils and sorbitol (SorbitoGlyceride, Gattefosse), pentaerythrityl tetraisostearate (Crodamol PTIS, Croda), pentaerythrityl distearate (Albunol DS, Taiwan Surf), pentaerythrityl tetraoleate (Liponate PO-4, Lipo Chem.), pentaerythrityl tetrastearate (Liponate PS-4, Lipo Chem.), pentaerythrityl tetracaprylate tetracaprate (Liponate PE-810, Lipo Chem.), and pentaerythrityl tetraoctanoate (Nilol Pentarate 408, NikLo). Also included as oils, are oil-soluble vitamins, such as vitamins A, D, E, K, etc., and derivatives thereof, such as tocopheryl PEG-1000 succinate (TPGS, available from Eastman).

Suitable polyglycerized fatty acids include, but are not limited to, polyglyceryl-2 stearate (NikLol DGMS, Nikko), polyglyceryl-2 oleate (Nildol DGMO, Nildco), polyglyceryl-2 isostearate (Nikkol DGMIS, Nikko), polyglyceryl-3 oleate (Caprol 3GO, ABITEC), polyglyceryl-4 oleate (Nildol Tetraglyn 1-O, NikLo), polyglyceryl 4 stearate (NikLol Tetraglyn 1-S, Niliko), polyglyceryl-6 oleate (Drewpol 6-1 O, Stepan), polyglyceryl-10 laurate (Nildcol Decaglyn 1-L, Nikko), polyglyceryl-10 oleate (NikLol Decaglyn 1-O, Nildo), polyglyceryl-10 stearate (Nikkol Decaglyn 1-S, Nikko), polyglyceryl-6 ricinoleate (Nikkol Hexaglyn PR-15, Nikko), polyglyceryl-10 linoleate (Nikkol Decaglyn 1-LN, Nikko), polyglyceryl-6 pentaoleate (Nikkol Hexaglyn 5-0, Nikko), polyglyceryl-3 dioleate (Cremophor G032, BASF), polyglyceryl-3 distearate (Cremophor GS32, BASF), polyglyceryl-4 pentaoleate (Nikkol Tetraglyn 5-0, Nikko), polyglyceryl-6 dioleate (Caprol( ) 6G20, ABITEC), polyglyceryl-2 dioleate (Nikkol DGDO, Nikko), polyglyceryl-10 trioleate (Nikkol Decaglyn 3-0, Nikko), polyglyceryl-10 pentaoleate (Nikkol Decaglyn 5-0, Nikko), polyglyceryl-10 septaoleate (Nikkol Decaglyn 7-0, Nikko), polyglyceryl-10 tetraoleate (Caprol 10G40, ABITEC), polyglyceryl-10 decaisostearate (Nikkol Decaglyn 10-IS, Nikko), polyglyceryl-101 decaoleate (Drewpol 10-10 O, Stepan), polyglyceryl-10 mono, dioleate (Caprol PGE 860, ABITEC), and polyglyceryl polyricinoleate (Polymuls, Henkel).

Suitable propylene glycol fatty acid esters include, but are not limited to, propylene glycol monocaprylate (Capryol 90, Gattefosse), propylene glycol monolaurate (Lauroglycol 90, Gattefosse), propylene glycol oleate (Lutrol OP2000, BASF), propylene glycol myristate (Mirpyl), propylene glycol monostearate (LIPO PGMS, Lipo Chem.), propylene glycol hydroxystearate, propylene glycol ricinoleate (PROPYMULS, Henkel), propylene glycol isostearate, propylene glycol monooleate (Myverol P-06, Eastman), propylene glycol dicaprylate dicaprate (Captex 200, ABITEC), propylene glycol dioctanoate (Captex 800, ABITEC), propylene glycol caprylate caprate (LABRAFAC PG, Gattefosse), propylene glycol dilaurate, propylene glycol distearate (Kessco PODS, Stepan), propylene glycol dicaprylate (Nikkol Sefsol 228, Nikko), and propylene glycol dicaprate (Nikkol PDD, Nikko).

Suitable mixtures of propylene glycol esters and glycerol esters include mixutes of oleic acid esters of propylene glycol (ATMOS 300, ARLACEL 186, ICI) or stearic acid esters of propylene glycol (ATMOS 150) and glycerol (ARLACEL 186).

Suitable mono- and diglycerides include, but are not limited to, monopalmitolein (C 16:1) (Larodan), monoelaidin (C 18:1) (Larodan), monocaproin (C6) (Larodan), monocaprylin (Larodan), monocaprin (Larodan), monolaurin (Larodan), glyceryl monomyristate (C14) (Nilol MGM, Nikko), glyceryl monooleate (C18:1) (PECEOL, Gattefosse), glyceryl monooleate (Myverol, Eastman), glycerol monooleate/linoleate (OLICINE, Gattefosse), glycerol monolinoleate (Maisine, Gattefosse), glyceryl ricinoleate (Softigen 701, HuIs), glyceryl monolaurate (ALDO MLD, Lonza), glycerol monopalmitate (Emalex GMS-P, Nihon), glycerol monostearate (Capmul GMS, ABITEC), glyceryl mono- and dioleate (Capmul GMO-K, ABITEC), glyceryl palmitic/stearic (CUTINA MD-A, ESTAGEL-G18), glyceryl acetate (Lamegin EE, Grunau GmbH), glyceryl laurate (Imwitor 312, HuIs), glyceryl citrate/lactate/oleate/linoleate (Imwitor) 375, HuIs), glyceryl caprylate (Imwitor 308, HuIs), glyceryl caprylate/caprate (Capmul MCM, ABITEC), caprylic acid mono- and diglycerides (Imwitor 988, HuIs), caprylic/capric glycerides (Imwitor 742, HuIs), mono- and diacetylated monoglycerides (Myvacet 9-45, Eastman), glyceryl monostearate (Aldo MS, Arlacel 129, ICI), lactic acid esters of mono and diglycerides (LAMEGIN GLP, Henkel), dicaproin (C6) (Larodan), dicaprin (ClO) (Larodan), dioctanoin (C8) (Larodan), dimyristin (C14) (Larodan), dipalmitin (C16) (Larodan), distearin (Larodan), glyceryl dilaurate (C 12) (Capsule GDL, ABITEC), glyceryl dioleate (Capmul( ) GDO, ABITEC), glycerol esters of fatty acids (GELUCIRE 39/01, Gattefosse), dipalmitolein (C16:1) (Larodan), 1,2 and 1,3-diolein (C18:1) (Larodan), dielaidin (C18:1) (Larodan), and dilinolein (C 18:2) (Larodan).

Suitable sterols and sterol derivatives include, but are not limited to, cholesterol, sitosterol, lanosterol, PEG-24 cholesterol ether (Solulan C-24, Amerchol), PEG-30 cholestanol (Phytosterol GENEROL series, Henkel), PEG-25 phytosterol (Nilol BPSH 25, Nikko), PEG-5 soyasterol (Nikkol BPS-5, NiIo), PEG-10 soyasterol (NikLol BPS-10, Niliko), PEG-20 soyasterol (Nikkol BPS-20, NikLo), and PEG-30 soyasterol (NikLol BPS-30, NikLo).

Suitable polyethylene glycol sorbitan fatty acid esters include, but are not limited to, PEG-10 sorbitan laurate (Liposorb L-10, Lipo Chem.), PEG-20 sorbitan monolaurate (Tween 20, Atlas/ICI), PEG-4 sorbitan monolaurate (Tween) 21, Atlas/ICI), PEG-80 sorbitan monolaurate (Hodag PSML-80, Calgene), PEG-6 sorbitan monolaurate (NikLol GL-I, NikLo), PEG-20 sorbitan monopalmitate (Tween 40, Atlas/ICI), PEG-20 sorbitan monostearate (Tween 60, Atlas/ICI), PEG-4 sorbitan monostearate (Tween (D 61, Atlas/ICI), PEG-8 sorbitan monostearate (DACOL MS S. Condea), PEG-6 sorbitan monostearate (Nikkol TS 106, NiIo), PEG-20 sorbitan tristearate (Tween 65, Atlas/ICI), PEG-6 sorbitan tetrastearate (NikLol OS-6, Nildco), PEG-60 sorbitan tetrastearate (NikLol GS-460, Nikko), PEG-5 sorbitan monooleate (Tweed 81, Atlas/ICI), PEG-6 sorbitan monooleate (Nikkol TO 106, Nikko), PEG-20 sorbitan monooleate (Tweedy 80, Atlas/ICI), PEG-40 sorbitan oleate (Emalex ET S040, Nihon Emulsion), PEG-20 sorbitan trioleate (Tweedy 85, Atlas/ICI), PEG-6 sorbitan tetraoleate (Nikkol GO-4, Nikko), PEG-30 sorbitan tetraoleate (Nikkol GO-430, Nikko), PEG-40 sorbitan tetraoleate (Nikkol GO-440, Nikko), PEG-20 sorbitan monoisostearate (Tween 120, Atlas/ICI), PEG sorbitol hexaoleate (Atlas G-1086, ICI), polysorbate 80 (Tweed 80, Pharma), polysorbate 85 (Tweed 85, Pharma), polysorbate 20 (Tween 20, Pharma), polysorbate 40 (Tween 40, Pharma), polysorbate 60 (Tween 60, Pharma), and PEG-6 sorbitol hexastearate (Nikkol OS-6, Nikko).

Suitable polyethylene glycol alkyl ethers include, but are not limited to, PEG-2 oleyl ether, oleth-2 (Brij 92/93, Atlas/ICI), PEG-3 oleyl ether, oleth-3 (Volpo 3, Croda), PEG-5 oleyl ether, oleth-5 (Volpo 5, Croda), PEG-10 oleyl ether, oleth-10 (Volpo 10, Croda), PEG-20 oleyl ether, oleth-20 (Volpo 20, Croda), PEG-4 lauryl ether, laureth-4 (Brij 30, Atlas/ICI), PEG-9 lauryl ether, PEG-23 lauryl ether, laureth-23 (Brij 35, Atlas/ICI), PEG-2 cetyl ether (Brij 52, ICI), PEG-10 cetyl ether (Brij 56, ICI), PEG-20 cetyl ether (BriJ 58, ICI), PEG-2 stearyl ether (Brij 72, ICI), PEG-10 stearyl ether (Brij 76, ICI), PEG-20 stearyl ether (Brij 78, ICI), and PEG-100 stearyl ether (Brij 700, ICI).

Suitable sugar esters include, but are not limited to, sucrose distearate (SUCRO ESTER 7, Gattefosse), sucrose distearate/monostearate (SUCRO ESTER 11, Gattefosse), sucrose dipalmitate, sucrose monostearate (Crodesta F-160, Croda), sucrose monopalmitate (SUCRO ESTER 15, Gattefosse), and sucrose monolaurate (Saccharose monolaurate 1695, Mitsubisbi-Kasei).

Suitable polyethylene glycol alkyl phenols include, but are not limited to, PEG-10-100 nonylphenol series (Triton X series, Rohm & Haas) and PEG-15-100 octylphenol ether series (Triton N-series, Rohm & Haas).

Suitable polyoxyethylene-polyoxypropylene block copolymers (“poloxamers”) include, but are not limited to, those of the following formula: HO(C₂H₄O)ₐ(C₃H₆O)_{b}(C₂H₄O)ₐH where “a” and “b” denote the number of polyoxyethylene and polyoxypropylene units, respectively. These copolymers are available in molecular weights ranging from 1000 to 15000 daltons, and with ethylene oxide/propylene oxide ratios between 0.1 and 0.8 by weight. Poloxamers are available, for example, under the tradenames Synperonic PE series (ICI), Pluronic series (BASF), Lutrol (BASF), Supronic, Monolan, Pluracare, and Plurodac.

Suitable polyoxyethylenes include, but are not limited to, PEG 300, PEG 400, and PEG 600.

Suitable sorbitan fatty acid esters include, but are not limited to, sorbitan monolaurate (Span-20, Atlas/ICI), sorbitan monopalmitate (Span-40, Atlas/ICI), sorbitan monooleate (Span-80, Atlas/ICI), sorbitan monostearate (Span-60, Atlas/ICI), sorbitan trioleate (Span-85, Atlas/ICI), sorbitan sesquioleate (Arlacel-C, ICI), sorbitan tristearate (Span-65, Atlas/ICI), sorbitan monoisostearate (Crill 6, Croda), and sorbitan sesquistearate (Nildcol SS-15, Nikko).

Suitable lower alcohol (C₂ to C₄) fatty acid esters include, but are not limited to, ethyl oleate (Crodamol HO, Croda), isopropyl myristate (Crodamol IPM, Croda), isopropyl palmitate (Crodamol IPP, Croda), ethyl linoleate (Nilol VF-E, Nikko), and isopropyl linoleate (NikLol VF-IP, Nikko).

Suitable ionic surfactants include, but are not limited to, sodium caproate, sodium caprylate, sodium caprate, sodium laurate, sodium myristate, sodium myristolate, sodium palmitate, sodium palmitoleate, sodium oleate, sodium ricinoleate, sodium linoleate, sodium linolenate, sodium stearate, sodium lauryl sulfate (dodecyl), sodium tetradecyl sulfate, sodium lauryl sarcosinate, sodium dioctyl sulfosuccinate, sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco chenodeoxycholate, sodium cholylsarcosinate, sodium N-methyl taurocholate, egg yolk phosphatides, hydrogenated soy lecithin, dimyristoyl lecithin, lecithin, hydroxylated lecithin, lysophosphatidylcholine, cardiolipin, sphingomyelin, phosphatidylcholine, phosphatidyl ethanolamine, phosphatidic acid, phosphatidyl glycerol, phosphatidyl serine, diethanolamine, phospholipids, polyoxy ethylene-10 oleyl ether phosphate, esterification products of fatty alcohols or fatty alcohol ethoxylates, with phosphoric acid or anhydride, ether carboxylates (by oxidation of terminal OH group of, fatty alcohol ethoxylates), succinylated monoglycerides, sodium stearyl fumarate, stearoyl propylene glycol hydrogen succinate, mono/diacetylated tartaric acid esters of mono- and diglycerides, citric acid esters of mono-, diglycerides, glyceryl-lacto esters of fatty acids, acyl lactylates, lactylic esters of fatty acids, sodium stearoyl-2-lactylate, sodium stearoyl lactylate, alginate salts, propylene glycol alginate, ethoxylated alkyl sulfates, alkyl benzene sulfones, alpha-olefin sulfonates, acyl isethionates, acyl taurates, alkyl glyceryl ether sulfonates, sodium octyl sulfosuccinate, sodium undecylenamido-MEA-sulfosuccinate, hexadecyl triammonium bromide, decyl trimethyl ammonium bromide, cetyl trimethyl ammonium bromide, dodecyl ammonium chloride, alkyl benzyldimethylammonium salts, diisobutyl phenoxyethoxydimethyl benzylammonium salts, alkylpyridinium salts, betaines (trialkylglycine), lauryl betaine (N-lauryl, N,N-dimethylglycine), and ethoxylated amines (polyoxyethylene-15 coconut amine). For simplicity, typical counter ions are provided above. It will be appreciated by one skilled in the art, however, that any bioacceptable counter ion can be used. For example, although the fatty acids are shown as sodium salts, other cationic counter ions can also be used, such as, for example, alkali metal cations or ammonium.

Suitable penetration enhancers include, but are not limited to, chloroform, methyl isobutyl ketone, monoethanolamine, tetradecylmethyl salfoxide, N-(2-Hydroxyethyl) pyrrolidone, dimethyl acetamide, tetrahydrofurfuryl alcohol, Clofibric acid amides, proteolytic enzymes, hexamethylene lauramide, terpenes and sesquiterpenes, alpha-bisbolol, d-limonene, and N,N-diethyl-m-toluamide.

Suitable thickening agents include, but are not limited to, agar, ammonium alginate, calcium alginate, colloidal silicon dioxide, dextrin, ethylcellulose, ethylene glycol palimtostearate, hydroxyethyl cellulose, hydroxyethylmethyl cellulose, hydroxypropyl cellulose, hydroxypropyl starch, hypromellose, methylcellulose, octyldodecanol, pectin, polycarbophil, polyethylene glycol, polyethylene oxide, potassium alginate, trehalose, xanthan gum, zinc stearate, acacia, alginic acid, bentonite, catbomers, carboxymethylcellulose calcium, carboxymethylcellulose sodium, carrageenan, certonia, cetostearyl alcohol, chitosan, cyclomethicone, gelatin, glycerin, glyceryl behenate, guar gum, hectorite, hydrogenated vegetable oil, magnesium aluminum silicate, maltodextrin, polydextrose, poly(methylvinyl ether/maleic anhydride), polyvinylacetate phthalate, polyvinyl alcohol, potassium chloride, povidone, propylene glycol alginate, saponite, soldium alginate, soldium chloride, stearyl alcohol, sucrose, sulfobutylether β-cyclodextrin, tragacanth, aluminum stearate, calcium silicate, glyceryl monooleate, glyceryl palmitostearate, polyethylene alkyl ethers, polymethacrylates, propylene carbonate, sodim ascorbate, sorbitol, anionic emulsifying wax, carnauba wax, cetyl alcohol, cetyl esters wax, dextrin, hydrogenated castor oil, microcrystalline wax, nonionic emulsifying wax, and paraffin.

Some additional examples of suitable excipients follow: ethylene glycol, propylene glycol, polypropylene glycol, butylene glycol, dipropylene glycol, ethylene glycol, hexylene glycol, and propylene glycol, ethyl hexanediol, propylene glycol monolaurate, propylene glycol monostearate, propylene glycol palmitostearate, propylene glycol ricinoleate, diisopropyl adipate, N-methyl-2-pyrrolidone, lactic acid, 1,2,6-hexanetriol, 1,3-dimethylol-5,5-dimethyl-hydantoin, 1-o-tolylbiguanide, 2-amino-2-methyl-1-propanol, 2-ethylhexyl salicylate, acetic acid, acetone, acrylates copolymer, acrylic acid homopolymer, acrylic acid/isooctylacrylate copolymer, adcote 72A103, adhesive tape, adhesive tape, aerotex resin 3730, ethyl alcohol, dehydrated ethyl alcohol, denatured ethyl alcohol, diluted 50% aqueous ethyl alcohol, alkyl ammonium sulfonic acid betaine, alkyl aryl sodium sulfonate, allantoin, almond oil, alpha-terpineol, alpha-tocopherol, aluminum acetate, aluminum chlorhydroxy allantoinate, aluminum hydroxide, aluminum hydroxide-sucrose, hydrated aluminum hydroxide gel, aluminum hydroxide gel F 500, aluminum hydroxide gel F 5000, aluminum monostearate, aluminum oxide, aluminum polyester, aluminum silicate, aluminum starch octenylsuccinate, aluminum stearate, aluminum sulfate anhydrous, amerchol C, amerchol-cab, ammonia solution, strong ammonia solution, ammonium hydroxide, ammonium lauryl sulfate, ammonium nonoxynol 4 sulfate, ammonium salt of C-12-C-15 linear primary alcohol ethoxylate, ammonyx, amphoteric-2, amphoteric-9, anhydrous dibasic calcium phosphate, anoxid SBN, antifoam, apricot kernel oil PEG-6 esters, aquaphor, arlacel, arlatone 289, ascorbic acid, ascorbyl palmitate, Canada balsam, beeswax, synthetic beeswax, beheneth-10, bentonite, bentonite, benzalkonium chloride, benzoic acid, benzyl alcohol, betadex, boric acid, butyl alcohol, butyl ester of PVM/MA copolymer, butyl stearate, butylated hydroxyanisole, butylated hydroxytoluene, butylene glycol, butylparaben, C20-40 pareth-24, calcium acetate, calcium chloride, calcium hydroxide, caprylic/capric triglyceride, caprylic/capric/stearic triglyceride, captan, caramel, carbomer 1342, carbomer 934, carbomer 934p, carbomer 940, carbomer 941, carbomer 974P, carbomer 980, carbomer 980, carbomer 981, carbomer 971, carbomer homopolymer type C, glycerin-based carbomer, carboxy vinyl copolymer, carboxymethylcellulose, carboxymethylcellulose sodium, carrageenan, carrageenan salt, castor oil, castor oil hydrogenated, cedar leaf oil, cellulose, cerasynt-se, ceresin, ceteareth-12, ceteareth-15, ceteareth-30, cetearyl alcohol, cetearyl alcohol/ceteareth-20, cetearyl octanoate, ceteth-10, ceteth-2, ceteth-20, ceteth-23, cetrimonium chloride, cetyl alcohol, cetyl esters, cetyl palmitate, cetylpyridinium chloride, chemoderm 640 IB, chlorobutanol, chlorocresol, chloroxylenol, cholesterol, choleth-24, citric acid, citric acid monohydrate, hydrous citric acid, cocamide diethanolamine, cocamide ether sulfate, cocamine oxide, coco betaine, cocoa butter, cocoglycerides, cocomonoethanolamide, coconut oil, fractioned coconut oil, cocoyl caprylocaprate, collagen, coloring suspension, cream base, creatinine, crospovidone, crospovidone, cyclomethicone, cyclomethicone/dimethicone copolyol, daubert 1-5 PESTR (matte) 164Z, dehydroacetic acid, dehymuls E, denatonium benzoate, dextrin, diazolidinylurea, dichlorobenzyl alcohol, dichlorodifluoromethane, dichlorotetrafluoroethane, diethanolamine, diethyl sebacate, diethylene glycol monoethyl ether, diethylene glycol monoethyl ether, dihydroxyaluminum aminoacetate, diisopropanolamine, diisopropyl adipate, diisopropyl dimerate, dimethicone 350, dimethicone 360, dimethicone copolyol, dimethicone MDX4-4210, dimethyl isosorbide, dimethyl sulfoxide, dioctyl phthalate, dipropylene glycol, disodium cocoamphodiacetate, disodium laureth sulfo succinate, disodium lauryl sulfosuccinate, docosanol, docusate sodium, duro-TAK 280-2516, duro-TAK 80-1196, duro-TAK 87-2070, duro-TAK 87-2194, duro-TAK 87-2287, duro-TAK 87-2296, duro-TAK 87-2888, duro-TAK 87-2979, edetate sodium, edetic acid, entsulfon, entsulfon sodium, essence, bouquet 9200, ethyl acetate, ethyl hexanediol, ethyl oleate, ethylcellulose, ethylene glycol, ethylene vinyl acetate copolymer, ethylenediamine, ethylenediamine dihydrochloride, ethylene-propylene copolymer, ethylparaben, eudragit E 100, fatty acid esters, fatty acid pentaerythriol ester, fatty acids, fatty alcohol citrate, ferric oxide, flavor rhodia pharmaceutical #RF 451, formaldehyde, formaldehyde solution, gelatin, gelva 737, gluconolactone, glycerin, glyceryl citrate, glyceryl isostearate, glyceryl laurate, glyceryl monostearate, glyceryl oleate, glyceryl oleate, glyceryl oleate/propylene glycol, glyceryl palmitate, glyceryl ricinoleate, glyceryl stearate-laureth-23, glyceryl stearate SE, glyceryl stearate/PEG-100 stearate, glyceryl stearate-stearamidoethyl diethylamine, glycol distearate, guar gum, hair conditioner (18N195-1M), herbacol, hexylene glycol, hyaluronate sodium, plasticized hydrocarbon gel, hydrochloric acid, diluted hydrochloric acid, hydrogen peroxide, hydrogenated palm/palm kernel oil PEG-6 esters, hydroxyethyl cellulose, hydroxyethyl cellulose, hydroxymethyl cellulose, hydroxyoctacosanyl hydroxystearate, hydroxypropyl cellulose, hydroxypropyl cellulose, hypromellose, imidurea, ink flexographic pink, ink/polyethylene, terephthalate/aluminum/polyethylene/sodium polymethacrylate/ethylene vinylacetate copolymer, irish moss extract, isoceteth-20, isooctylacrylate, isopropyl alcohol, isopropyl isostearate, isopropyl myristate, isopropyl myristate, isopropyl myristate-myristyl alcohol, isopropyl palmitate, isopropyl stearate, isostearic acid, isostearyl alcohol, jelene, kaolin, kathon CG, kathon CG II, lactate, lactic acid, dl-lactic acid, lactose, laneth, lanolin, lanolin alcohol-mineral oil, lanolin alcohols, acetylated lanolin alcohols, lanolin anhydrous, lanolin cholesterols, lanolin, hydrogenated, lauramine oxide, laurdimonium hydrolyzed animal collagen, laureth sulfate, laureth-2, laureth-23, laureth-4, lauric diethanolamide, lauric myristic diethanolamide, lauryl lactate, lauryl sulfate, lecithin, lemon oil, light mineral oil, limonene, dl-, lipocol SC-15, magnesium aluminum silicate, magnesium aluminum silicate hydrate, magnesium nitrate, magnesium stearate, mannitol, maprofix, medical adhesive modified S-15, medical antiform a-f emulsion, menthol, methoxypolyoxyethylene glycol 350, methyl alcohol, methyl gluceth-10, methyl gluceth-120 dioleate, methyl gluceth-20, methyl gluceth-20 sesquistearate, methyl glucose sesquistearate, methyl laurate, methyl salicylate, methyl stearate, methylcellulose, methylchloroisothiazolinone, ethylisothiazolinone, methylparaben, microcrystalline wax, mineral oil, multisterol extract, myristyl alcohol, myristyl lactate, n,n-bis(2-hydroxyethyl)stearamide, n,n-dimethyl lauramine oxide, n-3-chloroallyl-methenamine chloride, n-decyl-methyl sulfoxide, niacinamide, nitric acid, nonoxynol-15, nonoxynol-9, octadecene-1/maleic acid copolymer, octoxynol-1, octoxynol-9, octyl hydroxystearate, octyldodecanol, octyldodecanol, oleic acid, oleth-1O/oleth-5, oleth-2, oleth-20, oleyl alcohol, oleyl oleate, olive oil, orvus k liquid, palmitamine oxide, parabens, paraffin, white soft paraffin, parfum creme 45/3, peanut oil, pectin, PEG 6-32 stearate/glycol stearate, PEG-22 methyl ether/dodecyl glycol copolymer, PEG-25 propylene glycol stearate, PEG-45/dodecyl glycol copolymer, peglicol-5-oleate, pegoxol 7 stearate, pentadecalactone, pentaerythritol cocoate, peppermint oil, perfume 25677′, perfume bouquet, perfume E-1991, perfume GD 5604, perfume tana 90/42 SCBA, perfume W-1952-1, petrolatum, white petrolatum, petroleum distillates, phenonip, phenoxyethanol, phenylmercuric acetate, phosphoric acid, pine needle oil, plastibase-50w, polacrilin, poloxamer 124, poloxamer 181, poloxamer 182, poloxamer 188, poloxamer 237, poloxamer 407, polybutene, polycarbophil, polyester, fluoro-polyester chemical releasing agent, polyester fluorocarbon diacrylate, polyester polyamine copolymer, polyethylene, polyethylene glycol 1000, polyethylene glycol 1500, polyethylene glycol 1540, polyethylene glycol 200, polyethylene glycol 300, polyethylene glycol 300-1600, polyethylene glycol 3350, polyethylene glycol 400, polyethylene glycol 4000, polyethylene glycol 540, polyethylene glycol 600, polyethylene glycol 6000, polyethylene glycol 8000, polyethylene glycol 900, polyethylene terephthalates, polyhydroxyethyl methacrylate, polyisobutylene, polyisobutylene 1,200,000, polyisobutylene 1,200,000, polyisobutylene 35,000, polyisobutylene low molecular weight, polyisobutylene medium molecular weight, polyisobutylene/polybutene adhesive, polyoxyethylene-polyoxypropylene 1800, polyoxyethylene alcohols, polyoxyethylene fatty acid esters, polyoxyethylene propylene, polyoxyl 100 glyceryl stearate, polyoxyl 100 stearate, polyoxyl 15 cocamine, polyoxyl 150 distearate, polyoxyl 2 stearate, polyoxyl 20 cetostearyl ether, polyoxyl 4 dilaurate, polyoxyl 40 hydrogenated castor oil, polyoxyl 40 stearate, polyoxyl 400 stearate, polyoxyl 50 stearate, polyoxyl 6 and polyoxyl 32 palmitostearate, polyoxyl 6 isostearate, polyoxyl 60 hydrogenated castor oil, polyoxyl 75 lanolin, polyoxyl 8 laurate, polyoxyl 8 stearate, polyoxyl distearate, polyoxyl glyceryl stearate, polyoxyl lanolin, polyoxyl stearate, polypropylene, polyquaternium-1, polyquaternium-10, polyquaternium-7, polysorbate 20, polysorbate 20, polysorbate 40, polysorbate 60, polysorbate 65, polysorbate 80, polyvinyl acetate, polyvinyl alcohol, polyvinyl alcohol, polyvinyl chloride-polyvinyl acetate copolymer, polyvinylpyridine, potash, potassium citrate, potassium hydroxide, potassium soap, potassium sorbate, povidone acrylate copolymer, povidone hydrogel, povidone K29-32, povidone K90, povidone/eicosene copolymer, PPG-12/SMDI copolymer, PPG-15 stearyl ether, PPG-20 methyl glucose ether distearate, PPG-26 oleate, product WAT, promalgen type G, promulgen D, promulgen G, propellant A-46, propyl gallate, propylene carbonate, propylene glycol, propylene glycol, propylene glycol, propylene glycol diacetate, propylene glycol dicaprylate, propylene glycol monolaurate, propylene glycol monostearate, propylene glycol palmitostearate, propylene glycol ricinoleate, propylene glycol/diazolidinyl urea/methylparaben/propylparben, propylparaben, protein hydrolysate, quaternium-15, quaternium-52, quatrimycin hydrochloride, RA-2397, RA-3011, saccharin, saccharin sodium, safflower oil, scotchpak 1109, scotchpak 9739 backing film PET/EVA, SD alcohol 3A, SD alcohol 40, SD alcohol 40-2, SD alcohol 40b, silicon dioxide, colloidal silicon dioxide, silicone, silicone adhesive 4102, silicone emulsion, silicone/polyester film strip, simethicone, simethicone emulsion, sipon 1-20, sodium acetate, sodium acetate anhydrous, sodium alkyl sulfate, sodium benzoate, sodium cetearyl sulfate, sodium chloride, sodium chloride, sodium citrate, sodium citrate, sodium cocoyl sarcosinate, sodium dodecyl benzene sulfonate, sodium formaldehyde sulfoxylate, sodium hydroxide, sodium iodide, sodium lactate, sodium laureth sulfate, sodium laureth-2 sulfate, sodium laureth-5 sulfate, sodium lauroyl sarcosinate, sodium lauryl sulfate, sodium lauryl sulfoacetate, sodium metabisulfite, sodium phosphate, dibasic sodium phosphate, dibasic anhydrous sodium phosphate, dibasic dihydrate sodium phosphate, sodium phosphate, dibasic, heptahydrate monobasic sodium phosphate, monobasic sodium phosphate anhydrous, monohydrate sodium phosphate, monobasic, sodium polyacrylate, sodium pyrrolidone carboxylate, sodium sulfite, sodium sulfosuccinated undecyclenic monoalkylolamide, sodium thiosulfate, sodium xylenesulfonate, solulan, somay 44, sorbic acid sorbitan, sorbitan monolaurate, sorbitan monooleate, sorbitan monooleate, sorbitan monopalmitate, sorbitan monostearate, sorbitan sesquioleate, sorbitol, sorbitol solution, soybean flour, soybean oil, spearmint oil, spermaceti, squalane, starch, starch aluminum octenyl succinate, stearalkonium chloride, stearalkonium hectorite/propylene carbonate, stearamidoethyl diethylamine, steareth-10, steareth-100, steareth-2, steareth-20, steareth-21, stearic acid, stearoxytrimethylsilane, steartrimonium hydrolyzed animal collagen, stearyl alcohol, stearyl citrate, styrene/isoprene/styrene block copolymer, sucrose, sucrose distearate, sucrose polyesters, sulfacetamide sodium, sulfuric acid, surfactol SQ, talc, tall oil, tallow glycerides, tartaric acid, tenox, tenox-2, tert-butyl alcohol, thimerosal, titanium dioxide, titanium dioxide, tocopherol, tocophersolan, triacetin, trichloromonofluoromethane, trideceth-10, medium chain triglycerides, trihydroxystearin, trilaneth-4 phosphate, trilaureth-4 phosphate, trisodium citrate dihydrate, trisodium citrate, anhydrous, trisodium hedta, triton X-200 sodium salt of alkylauryl polyether sulfonate, trolamine, trolamine lauryl sulfate, tromethamine, tromethamine, tyloxapol, undecylenic acid, union 76 AMSCO-RES 6038, vegetable oil, hydrogenated vegetable oil, viscarin, viscose/cotton, wax, dehydag, emulsifying wax, white wax, wecobee FS, xanthan gum, xanthan gum and zinc acetate.

Other suitable pharmaceutically acceptable excipients include those listed in the Federal Food and Drug Administration's Inactive Ingredient Guide (updated through Dec. 31, 2010), the contents of which are herein incorporated by reference in their entirety.

In one embodiment, the pharmaceutically acceptable excipient is present in an amount of about 1% to about 99%, about 5% to about 80%, about 10% to about 70%, about 15% to about 60%, about 20% to about 50%, or about 40% to about 60% by weight of the composition. In another embodiment, the pharmaceutically acceptable excipient is present in an amount of about 1% to about 20%, about 2% to about 10%, about 1% to about 5%, about 2% to about 5%, about 5% to about 15%, or about 5% to about 10% by weight of the composition.

In another embodiment, the composition further comprises an adjuvant. Suitable adjuvants include, but are not limited to, antioxidants, preserving agents, stabilizing agents, wetting agents, thickening agents, emulsifying agents and the like. In other embodiments, the composition further comprises a solvent, an antioxidant, an emollient, a humectant, a preservative, an emulsifier, a pH agent, a film-forming agent, or a combination thereof.

Suitable solvents include acetone, hydrocarbons, glycols, polyurethanes, and others known in the art. Suitable emollients include mineral oil, propylene glycol dicaprylate, lower fatty acid esters, lower alkyl ethers of propylene glycol, cetyl alcohol, cetostearyl alcohol, stearyl alcohol, stearic acid, wax, and others known in the art.

Suitable antioxidants include sodium bisulfite, butylated hydroxytoluene, edetate disodium, benzyl alcohol, ascorbic acid, citric acid, malic acid, fumaric acid, lactic acid, and propionic acid, and mixtures thereof. In one embodiment, the antioxidant is sodium bisulfite, butylated hydroxytoluene, or edetate disodium, or a mixture thereof.

Suitable humectants include glycerin, sorbitol, and others known in the art.

Suitable emulsifiers include glyceryl monostearate, glyceryl monoleate, stearic acid, polyoxyethylene cetyl ether, polyoxyethylene cetostearyl ether, polyoxyethylene stearyl ether, polyethylene glycol stearate, propylene glycol stearate, and others known in the art.

Suitable pH agents include hydrochloric acid, phosphoric acid, diethanolamine, triethanolamine, sodium hydroxide, monobasic sodium phosphate, dibasic sodium phosphate, and others known in the art. Suitable pH agents also include organic acids, for example, of the formula CₙH₍₂ₙ₊₂₎COOH, (where n is an integer of 1 to 6). Suitable organic acids include, but are not limited to, acetic acid, citric acid, tartaric acid, fumaric acid, lactic, glycolic and other alpha hydroxy acids, malic acid, carnitine, glutamic acid, aspartic acid and others known in the art. In one embodiment, the organic acid is present in am amount of about 0.01 percent to about 15 percent by weight of the composition. In another embodiment, the organic acid is present in an amount of about 1 percent to about 15 percent by weight of the composition. In another embodiment, the organic acid is present in an amount of about 2 percent to about 5 percent by weight of the composition. In one embodiment, the organic acid is present in the composition in an amount sufficient to provide a pH of less than about 7. In another embodiment, the organic acid is present in the composition in an amount sufficient to provide a pH of less than 5. In another embodiment, the organic acid is present in the composition in an amount sufficient to provide a pH of less than about 4. In another embodiment, the organic acid is present in the composition in an amount sufficient to provide a pH of about 3 to about 4. In another embodiment, the organic acid is present in an amount sufficient to provide a pH of about 2.5 to about 3.5. In another embodiment, the organic acid is present in the composition in an amount sufficient to provide a pH of about 3.

Suitable preservatives include benzyl alcohol, sodium benzoate, parabens, and others known in the art.

Suitable film-forming agents include maleic anhydride/methyl vinyl ether copolymers such as Gantrez copolymers sold by Internationals Specialty Products (Wayne, NJ.), as well as the ethyl, isopropyl, and butyl esters of these copolymers, and maleic anhydride/butyl vinyl ether copolymers. Hydroxy alkylcellulose polymers, such as Krucel<(R)> hydroxypropyl cellulose sold by Hercules Incorporated (Wilmington, Del.) may also be used as film-forming agents.

In one embodiment, the composition does not include any grade of white or yellow petrolatum recognized in the art as suitable for human application. In another embodiment, the composition does not include material commercially available as Penreco Snow White Pet USP. In another embodiment, the composition does not include hydrocarbon mixtures formulated with mineral oils in combination with paraffin waxes of various melting points. In another embodiment, the composition does not include a lipophilic emollient selected from the group consisting of: petrolatum; and esters of fatty acids.

In some embodiments, the composition does not comprise an inorganic salt. In other embodiments, the composition does not comprise an antioxidant.

In one embodiment, the composition does not comprise water or ethanol. In some embodiments, the composition comprises less than about 15%, less than about 10%, less than about 9.5%, less than about 9%, less than about 8.5%, less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5%, less than about 12%, less than about 1.5%, less than about 1%, or less than about 0.5% by weight water. In other embodiments, the composition comprises less than about 15%, less than about 10%, less than about 9.5%, less than about 9%, less than about 8.5%, less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5%, less than about 12%, less than about 1.5%, less than about 1%, or less than about 0.5% by weight ethanol. In one embodiment, the composition does not comprise petrolatum. In some embodiments, the composition comprises less than about 15%, less than about 10%, less than about 9.5%, less than about 9%, less than about 8.5%, less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5%, less than about 12%, less than about 1.5%, less than about 1%, or less than about 0.5% by weight petrolatum. In one embodiment, the composition does not comprise acetone. In some embodiments, the composition comprises less than about 15%, less than about 10%, less than about 9.5%, less than about 9%, less than about 8.5%, less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5%, less than about 12%, less than about 1.5%, less than about 1%, or less than about 0.5% by weight acetone.

In one embodiment, the pH of the composition is less than about 7. In another embodiment, the pH of the composition is less than 5. In another embodiment, the pH of the composition is less than about 4. In another embodiment, the pH of the composition is about 3 to about 4. In another embodiment, the pH of the composition is about 2.5 to about 3.5. In another embodiment, the pH of the composition is about 3.

In one embodiment, the viscosity of the composition is more than the viscosity of water (about 1 cps) and less than the viscosity of petrolatum (about 64,000 cps). In another embodiment, the viscosity of the composition is about 5,000 cps to about 50,000 cps. In another embodiment, the viscosity of the composition is about 15,000 cps to about 40,000 cps. In another embodiment, the viscosity of the composition is about 20,000 cps to about 35,000 cps.

In another embodiment, the viscosity of the composition is about 25,000 cps to about 35,000 cps. Viscosity can be measured with a Brookfield programmable rheometer, model RVDV-III with cone plate configuration using spindle CPE52, or equivalent apparatus. Viscosity measurements can be taken at 25° C. and 1 rpm over a period of 5-10 minutes, using a 0.5 mL sample size. In one embodiment, the composition is in the form of a non-Newtonion fluid, i.e., the viscosity of the composition varies with the forces acting on the composition (such as shear rate).

In one embodiment, the composition is in the form of a single phase gel. Typically, the single phase gel consists of one or more organic macromolecules uniformly distributed throughout a liquid with no apparent boundary between the dispersed macromolecule and liquid.

In another embodiment, the composition is in the form of a solution. In another embodiment, the composition is in the form of a non-aqueous solution.

C. Stability

In some embodiments, the nitrogen mustard alkylating agents disclosed herein are bifunctional alkylators, i.e., have two arms terminated with chlorine (“CR₂CR₂Cl”). When one arm terminated with chlorine is absent, the nitrogen mustard alkylating agent is referred to as a monofunctional alkylator or a “half-mustard.”

It is believed that nucleophiles in the composition or in the environment may degrade the nitrogen mustard alkylating agent to form a nitrogen mustard degradation product by reacting with the nitrogen mustard to displace one or more terminal chlorides of the nitrogen mustard by nucleophilic substitution.

Nucleophiles are defined as molecules having electron-rich functional groups (“E”), such as —O—, —NH—, or —S—. The most nucleophilic nucleophiles are believed to be water or nucleophiles having the electron-rich functional group covalently bonded to a primary carbon atom, such as methanol or ethanol. Nucleophiles include any pharmaceutically acceptable excipient having an electron-rich functional group (E) known to the skilled artisan. Some such pharmaceutically acceptable excipients are described, for example, in the HANDBOOK OF PHARMACEUTICAL EXCIPIENTS (5th ed., 2006, R. C. Rowe, et al., eds.), the contents of which are herein incorporated by reference.

The degradation of a nitrogen mustard alkylating agent by a nucleophile having an electron-rich functional group is illustrated, for example, by Reactions 2a and 2b below:

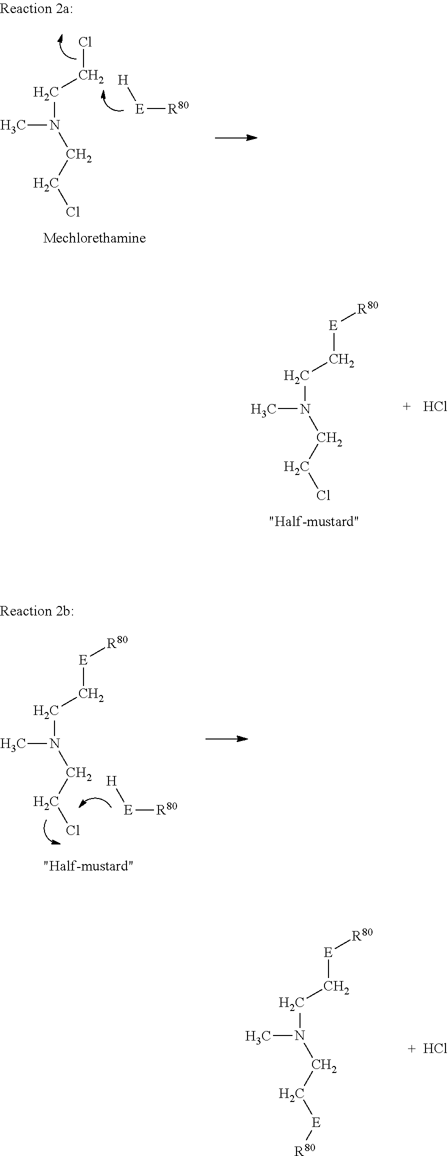

wherein each R⁸⁰ is independently a linear or branched alkyl group having 1-12 carbon atoms that is optionally substituted with one or more —COOH or —OH, and that is optionally interrupted by one or more —O—, —N—, —(CO)—, or —O—(CO)—. As used herein, the term “interrupted,” when referring to an alkyl group, means that one or more of the carbon-carbon bonds of the alkyl group is replaced with a —O—, —N—, —(CO)—, or —O—(CO)—, for example, as follows:

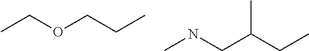

C₅ linear alkyl group interrupted by a —O—; C₆ branched alkyl group interrupted by a —N—; or

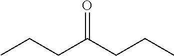

C₇ linear alkyl group interrupted by a —(CO)—.

In one embodiment, the composition is stable, i.e., at least about 80% of the alkylating agent is present in the composition or less than about 20% by weight degradation product of the alkylating agent is present in the composition after storage. In one embodiment, the composition is stored at a temperature of at least about −20° C. In another embodiment, the composition is stored at a temperature of about −20° C. to about −10° C. In one embodiment, the composition is stored at a temperature of at least about 2° C. In another embodiment, the composition is stored at a temperature of about 2° C. to about 8° C. In another embodiment, the composition is stored at room temperature. In another embodiment, the composition is stored at about 25° C. In another embodiment, the composition is stored for about 3 months to about 3 years.

In one embodiment, at least about 80% of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, at least about 85% of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, at least about 90% of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, at least about 95% of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, at least about 98% of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, at least about 99% of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months.

In one embodiment, less than about 20% by weight degradation product of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, less than about 15% by weight degradation product of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, less than about 10% by weight degradation product of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, less than about 5% by weight degradation product of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, less than about 1% by weight degradation product of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, less than about 0.5% by weight degradation product of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months.

In one embodiment, the composition is stored in a glass vial sealed from the atmosphere. In another embodiment, the composition is stored in an amber vial sealed from the atmosphere. In another embodiment, the composition is stored in an aluminum foil-lined container. In another embodiment, the composition is stored in an aluminum foil tube.

In another embodiment, the composition is stored in a plastic container. In another embodiment, the composition is stored in a polypropylene container.

In one embodiment, the composition is stored under standard storage conditions (i.e., about 25° C. and about 60% relative humidity). In one embodiment, the composition is stored under accelerated storage conditions (i.e., about 40° C. and about 75% relative humidity).

In one embodiment, the composition is stable in the presence of water. In another embodiment, the composition is stable and comprises 1%, 2%, 5%, 10%, 15%, or 20% by weight water.

In one embodiment, the alkylating agent is a nitrogen mustard and the degradation product is a nitrogen mustard degradation product.

In some embodiments, the nitrogen mustard degradation product is a half-mustard. In some embodiments, the half-mustard has the following structure (DP-A) or (DP-B):

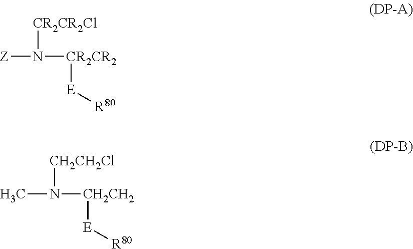

wherein:

Z is a linear alkyl group having 1-6 carbon atoms;

each R is independently hydrogen or a linear alkyl group having 1-6 carbon atoms;

each E is independently —O—, —NH—, or —S—; and

each R⁸⁰ is independently a linear or branched alkyl group having 1-12 carbon atoms that is optionally substituted with one or more —COOH or —OH, and that is optionally interrupted by one or more —O—, —N—, —(CO)—, or —O—(CO)—.

In some embodiments, the moiety E-R⁸⁰ is

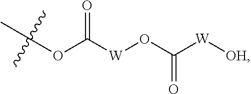

wherein W is a linear or branched alkyl group having 1-6 carbon atoms that is optionally substituted with —COOH. In another embodiment, the moiety E-R⁸⁰ is

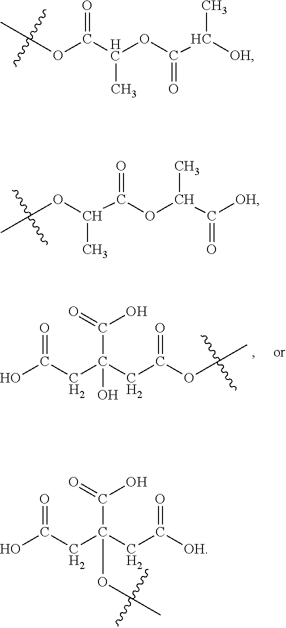 [SMILES: *OC(=O)C(C)OC(=O)C(C)O ; *OC(C)C(=O)OC(C)C(=O)O ; *OC(=O)CC(O)(CC(=O)O)C(=O)O ; *OC(CC(=O)O)(CC(=O)O)C(=O)O]

In one embodiment, the moiety E-R⁸⁰ is

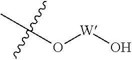

wherein W′ is a linear or branched alkyl group having 1-6 carbon atoms. In another embodiment, the moiety E-R⁸⁰ is

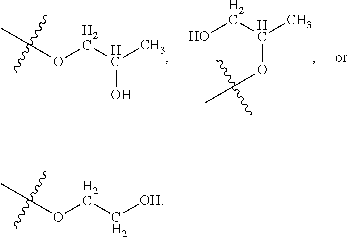

In other embodiments, the half-mustard has the structure (DP-A) or (DP-B),

wherein Z is a linear alkyl group having 1-6 carbon atoms; each E is independently —O—, —NH—, —S—; —OC(O)CH(CH₃)OC(O)CH(CH₃)—; —OCH(CH₃)C(O)OCH(CH₃)—; or —O(CH₂)₂—O—(CH₂)₂O—; and each R⁸⁰ is independently a linear or branched alkyl group having 1-12 carbon atoms, —COOH, or —OH.

In other embodiments, the nitrogen mustard degradation product has the following structure (DP-C) or (DP-D):

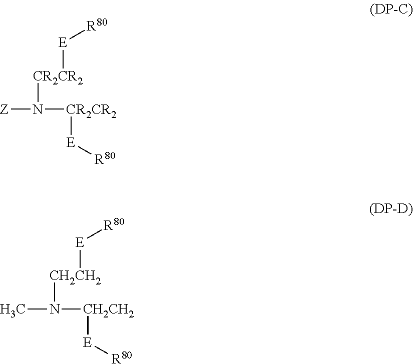

wherein:

Z is a linear alkyl group having 1-6 carbon atoms;

each R is independently hydrogen or a linear alkyl group having 1-6 carbon atoms;

each E is independently —O—, —NH—, or —S—; and

each R⁸⁰ is independently a linear or branched alkyl group having 1-12 carbon atoms that is optionally substituted with one or more —COOH or —OH, and that is optionally interrupted by one or more —O—, —N—, —(CO)—, or —O—(CO)—.

In some embodiments, each E-R⁸⁰ moiety is independently

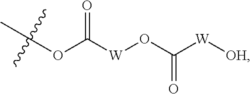

wherein W is a linear or branched alkyl group having 1-6 carbon atoms that is optionally substituted with —COOH. In another embodiment, the each E-R⁸⁰ moiety is independently

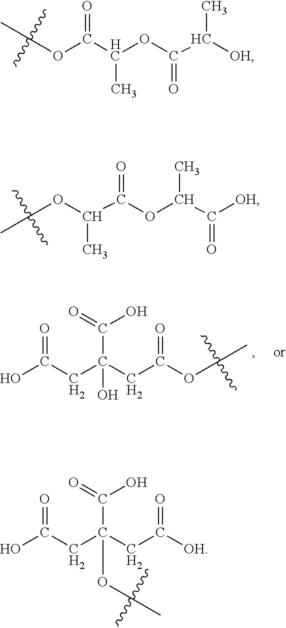 [SMILES: *OC(=O)C(C)OC(=O)C(C)O ; *OC(C)C(=O)OC(C)C(=O)O ; *OC(=O)CC(O)(CC(=O)O)C(=O)O ; *OC(CC(=O)O)(CC(=O)O)C(=O)O]

In one embodiment, each E-R⁸⁰ moiety is independently

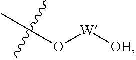

wherein W′ is a linear or branched alkyl group having 1-6 carbon atoms. In another embodiment, each E-R⁸⁰ moiety is independently

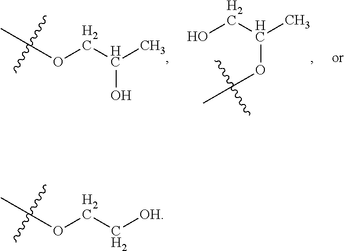

In other embodiments, nitrogen mustard degradation product has the structure (DP-C) or (DP-D), wherein Z is a linear alkyl group having 1-6 carbon atoms; each E is independently —O—, —NH—, —S—; —OC(O)CH(CH₃)OC(O)CH(CH₃)—; —OCH(CH₃)C(O)OCH(CH₃)—; or —O(CH₂)₂—O—(CH₂)₂O—; and each R⁸⁰ is independently a linear or branched alkyl group having 1-12 carbon atoms, —COOH, or —OH.

In one embodiment, the composition has a duration of activity from about 3 months to about 3 years.

### III. METHODS FOR TREATING SKIN DISORDERS

In one embodiment, the invention encompasses methods for treating a skin disorder comprising topically administering to a subject in need thereof a stable composition comprising an effective amount of an alkylating agent or a pharmaceutically acceptable salt, solvate, or prodrug thereof, and at least one pharmaceutically acceptable excipient.

In one embodiment, the alkylating agent is a nitrogen mustard. In another embodiment, the alkylating agent is a nitrogen mustard of Structure (VII), (VIII), (IX), (X), (XII), (XIII), (XIV), (XV), (XVI), (XVII), (XVIII) or (XIX). In another embodiment, the nitrogen mustard is bis-(2-chloroethyl)ethylamine, bis-(2-chloroethyl)methylamine, or tris-(2-chloroethyl)amine. In another embodiment, the nitrogen mustard is bis-(2-chloroethyl)methylamine.

In one embodiment, the skin disorder is a T-cell mediated skin disorder. In one embodiment, the T-cell mediated skin disorder is psoriasis, actinic keratosis, cutaneous T-cell lymphoma, cutaneous B-cell lymphoma, mycosis fungoides, alopecia, alopecia greata, or vitiligo.

In one embodiment, the skin disorder is psoriasis, eczema, actinic keratosis, lupus, sarcoidosis, alopecia, alopecia greata, cutaneous T-Cell lymphoma, i.e., mycosis fungoides, lymphoreticular neoplasia, pleural or peritoneal effusions, cutaneous B-cell lymphoma, pseudolymphoma of the skin, squamous cell carcinoma, basal cell carcinoma, bronchogenic carcinoma, malignant melanoma, lymphosarcoma, chronic lymphocytic leukemia, polycythemia vera, lymphomatoid papulosis, Mucha-Habberman's disease (PLEVA), or vitiligo.

In one embodiment, the composition of alkylating agent is topically administered to humans or animals in the form of a sterile solution or suspension that contains a suitable quantity of alkylating agent. In one embodiment, the composition comprises an effective amount of alkylating agent. In some embodiments, the topical solution or suspension is incorporated in a slow release non-aqueous matrix for administering transdermally.

In one embodiment, the composition is topically administered to the subject once daily. In another embodiment, the composition is topically administered to the subject twice daily. In another embodiment, the composition is topically administered to the subject every other day, every third day, every fourth day, every fifth day, every sixth day, or once weekly.

In some embodiments, the effective amount of alkylating agent is about 1 ng to about 40 mg per 1.9 m² per day, about 10 ng to about 10 mg per 1.9 m² per day, or about 100 ng to about 4 mg per 1.9 m² per day. In other embodiments, the effective amount of alkylating agent is about 0.5 ng to about 20 mg per m² per day, about 5 ng to about 5 mg per m² per day, or about 50 ng to about 2 mg per m² per day.

In other embodiments, the effective amount of alkylating agent is about 1 ng to about 40 mg per 60 kg per day, about 10 ng to about 10 mg per 60 kg per day, or about 100 ng to about 4 mg per 60 kg per day. In other embodiments, the effective amount of alkylating agent is about 0.02 ng to about 0.7 mg per kg per day, about 0.2 ng to about 0.2 mg per kg per day, or about 1.7 ng to about 0.07 mg per kg per day.

In some embodiments of the methods, the composition contains a vehicle or carrier that ameliorates skin irritation that can result from administration of the nitrogen mustard, pharmaceutically acceptable salt of the nitrogen mustard, or nitrogen mustard prodrug. In some embodiments, the composition is effective to treat the skin disorder, but does not cause hypersensitivity reactions.

In another embodiment, the compositions of the invention can be used as adjunct therapy in combination with existing therapies, such as for hyperthermia or in the management of cancer treatment in patients having cancer. In one embodiment, the invention encompasses a method for treating a T-cell mediated skin disorder comprising administering a nitrogen mustard and another therapeutic agent. In one embodiment, the other therapeutic agent is a steroid. Suitable steroids include, but are not limited to, betamethasone, clobetasol, fluocinonide, halobetasol, desoximetasone, diflorasone, halocinonide, triamcinolone, amcinonide, flurandrenolide, fluticasone, mometasone, desonide, hydrocortisone, prednicarbate, alclometasone, clocortolone, amcinonide, fluocinonlone, clobetasone, desonide, and bexarotine.

In some embodiments, topical administration of a composition of the invention is effective to produce a response to treatment. In one embodiment, response to treatment is determined based on a controlled trial. Typically, the controlled trial is an observer-blinded trial. The observer-blinded trial may be performed with or without biases.

Response to treatment can be measured using a Composite Assessment of Index Lesion Score (“CAILS”). The CAILS is determined by: (a) assessing a severity score for each of the following symptoms for up to 5 index lesions: erythemia (0=none to 8=severe), scaling (0=none to 8=severe), plaque elevation (0=none to 3=severe), hypo/hyperpigmentation, and surface area (0=none to 9=severe); and (b) adding the severity score for each symptom to obtain a CAILS. The maximum CAILS is 140 and the minimum is 0. Response is determined when the CAILS at the end of treatment is greater than or equal to 50% lower than the CAILS at baseline.

Response to treatment can also be measured using a Severity Weighted Assessment Tool (“SWAT”) score, improvement in the percent of total body surface area, the time to response, or the time to disease progression. The SWAT score is determined by measuring each lesion as a percentage of total body surface area, and multiplying it by a severity-weighting factor (1=patch, 2=plaque, 3=tumor). Response is determined when the SWAT score at the end of treatment was greater than or equal to 50% lower than the SWAT score at baseline.

In some embodiments, the response rate in a group of human patients is greater than about 60% after at least six months of treatment with a composition of the invention. In other embodiments, the response rate in a group of human patients is greater than about 65%, greater than about 70%, greater than about 75%, greater than about 80%, greater than about 85%, greater than about 90%, or greater than about 95% after at least six months of treatment with a composition of the invention.

In other embodiments, the response rate in an intent-to-treat group of human patients is greater than about 50% after treatment with a composition of the invention. In other embodiments, the response rate in an intent-to-treat group of human patients is greater than about 55%, greater than about 60%, greater than about 65%, greater than about 70%, greater than about 75%, greater than about 80%, greater than about 85%, greater than about 90%, or greater than about 95% after treatment with a composition of the invention.

In other embodiments, the response rate in a group of human patients is greater than about 55% upon two months of treatment. In other embodiments, the response rate in a group of human patients is greater than about 60%, greater than about 65%, greater than about 70%, greater than about 75%, greater than about 80%, greater than about 85%, greater than about 90%, or greater than about 95% upon two months of treatment with a composition of the invention.

In some embodiments, the time to achieve a response rate of 50% in a group of human patients is about 40 weeks or less. In other embodiments, the time to achieve a response rate of 50% in a group of human patients is about 35, about 30, about 25, about 20, about 15, about 10, or about 5 weeks or less.

In some embodiments, topical administration of a composition of the invention is more effective in producing a response to treatment than a reference composition consisting of an equivalent amount of the nitrogen mustard, a hydrophobic carrier, and an organic solvent. Typically, the reference composition consists of a nitrogen mustard in aquaphor (i.e., petrolatum, mineral oil, ceresin, lanolin, panthenol, glycerin, bisabolol, and ethanol).

In one embodiment, the response rate achieved in a group of human patients upon application of a composition of the invention is greater than the response rate achieved upon application of the reference composition. In another embodiment, the response rate upon application of a composition of the invention is at least about 5%, at least about 10%, at least about 15%, at least about 20%, at least about 25%, at least about 30%, at least about 35%, or at least about 40% greater than the response rate achieved upon application of the reference composition.

In another embodiment, the time to achieve a response rate of 50% in a group of human patients upon application of a composition of the invention is less than the time to achieve a response rate of 50% upon application of the reference composition. In another embodiment, the time to achieve a response rate of 50% in a group of human patients is at least about 10%, at least about 15%, at least about 20%, at least about 25%, at least about 30%, at least about 35%, or at least about 40% less than the time to achieve a response rate of 50% achieved upon application of the reference composition. In some embodiments, the time to achieve a response rate of 50% in a group of human patients is at least about 5, at least about 10, at least about 15, or at least about 20 weeks less than the time to achieve a response rate of 50% achieved upon application of the reference composition.

Typically, the safety of the compositions of the invention upon application is comparable to the safety observed upon application of the reference composition. In one embodiment, the percent occurrence of an adverse event in a group of human patients upon application of the composition is substantially equal to the percent occurrence of the adverse event upon application of the reference composition. Adverse events include, but are not limited to, skin toxicity, such as allergic contact dermatitis, irritant contact dermatitis, or hypersensitivity, or skin cancer. In one embodiment, a greater response rate is observed in patients who experience more adverse events.

### IV. OTHER USES

The compositions of the invention have a number of additional uses and applications, such as formulation aids and as concentrated sources of alkylating agents for dilution and incorporation into a variety of dispersed systems and pharmaceutical products.

In one embodiment, the invention encompasses methods of using the above-described compositions as a formulation aid, as and as a means of storing, transporting, and dispensing discrete quantities of an alkylating agent for use in pharmaceutical formulations and other preparations. In one embodiment, the composition comprises an alkylating agent dispersed in 2-(2-ethoxyethoxy) ethanol.

In another embodiment, the invention encompasses an alkylating agent or agents dispersed in 2-(2-ethoxyethoxy) ethanol for use as a formulation aid, where said formulation aid is employed as a dispersion of a pharmaceutically acceptable alkylating agent or mixture of alkylating agents for subsequent dispersion and dilution into a bulk pharmaceutical product during the formulation and manufacture of said product.

As a formulation aid, the composition can serve as a pre-solvated, pre-dispersed form of an alkylating agent for ready dispersion and homogeneous mixing into a pharmaceutical formulation or other preparation, such as a solution, a suspension, an ointment, a cream, a lotion, a plaster, a spray, a colloid and a paste. Such a pre-dispersed form of an alkylating agent, already de-gassed and solvated, facilitates homogeneous mixing into such dosage forms while minimizing or eliminating clumping, flocculation, agglomeration, sticking and caking of alkylating agents.

The composition can be stored in any suitable container, such as a jar, a bottle, a flask, a bag, a collapsible bag, a bladder, a syringe, a collapsible tube or a drum. Said container might also have an appropriate dispensing port, such as a mouth, a spigot, a valve, a syringe port, and a pump. Said container might also be pressurized, or be charged by or attached to an inert gas source, such as dry nitrogen or helium, in order to further maintain stability of the dispersion and replace the dispensed volume of the dispersion with inert gas.

In another embodiment, the invention encompasses a method of formulating a pharmaceutical product, a component of which is at least one hydrolytically unstable alkylating agent(s), comprising: providing a formulation aid, wherein said formulation aid is a pre-solvated or pre-dispersed form of the alkylating agent; and dispersing the formulation aid into a pharmaceutical formulation or other preparation, wherein the formulation aid and the pharmaceutical formulation are substantially homogeneous. In one embodiment, the alkylating agent is a nitrogen mustard. In one embodiment, the formulation aid is 2-(2-ethoxyethoxy)ethanol.

In another embodiment, the invention encompasses a method for preparing a composition comprising an alkylating agent or a pharmaceutically acceptable salt, solvate, or prodrug thereof, and a pharmaceutically acceptable excipient, comprising: combining the alkylating agent or pharmaceutically acceptable salt, solvate, or prodrug thereof and the pharmaceutically acceptable excipient.

### EXAMPLES

### Example 1

### Identifying and Quantifying Nitrogen Mustard Degradation Products in MCHCl Ointments

Mechlorethamine HCl ointment according to Composition D (Table 1) was prepared and stored under various storage conditions.

TABLE 1
Composition D

| | Quality | Percent by weight |
|---|---|---|
| Component | Standard | of the composition |
| Mechlorethamine hydrochloride | USP | 0.001-5% |
| Hydroxypropyl cellulose | NF | 0.01-5% |
| Edetate disodium (dihydrate) | USP | 0.01-1% |
| (DL) Menthol | USP | 0.01-1% |
| Butylated hydroxytoluene | NF | 0.01-10% |
| 2-(2-ethoxyethoxy)ethanol) | NF | 1-99% |
| Isopropyl alcohol | USP | 1-50% |
| Propylene glycol | USP | 1-50% |
| Glycerin | USP | 1-50% |
| Lactic acid (racemic) | USP | 1-25% |
| Sodium chloride | USP | 0.01-10% |
| Total | | 100% |

The degradation of the mechlorethamine HCl was measured over time and the degradation products identified and quantified by HPLC/MS (Table 2).

TABLE 2
HPLC-MS Parameters

| Device: | Agilent HP1100 HPLC system equipped with | | |
|---|---|---|---|
| | diode array detector | | |
| | Micromass QTOF-API US mass spectromater | | |
| | MassLynx 4.0 with SP 4 | | |
| | Micromass QTOF-Ultima mass spectromater | | |
| | MassLynx 4.0 with SP 4 | | |
| Column: | Water Symmetry ® C18 column, 3.5μ, 100Å, | | |
| | 150 × 2.1 mm | | |
| Column Temp.: | 25° C. | | |
| Flow rate: | 0.2 mL/minute | | |
| Run time: | 60 minutes | | |
| Cone voltage: | 35 V, 75 V | | |
| Mobile phase: | A: 0.1% formic acid in water | | |
| | B: 0.1% formic acid in acetonitrile: water (95:5) | | |
| Gradient: | Time (minutes) | % A | % B |
| | 0 | 99% | 1% |
| | 3.10 | 1% | 99% |
| | 22 | 1% | 99% |
| | 41 | 99% | 1% |
| | 50 | 99% | 1% |
| | 60 | 99% | 1% |

The results are reported in Table 3 below and the structures of the identified degradation products are shown in Table 4 below.

TABLE 3

| | Storage Condition | | |
|---|---|---|---|
| | 9-12 months | 25-55 months | 1-2 months |
| | storage at 2-8° C. | storage at 2-8° C. | storage at 25° C. |
| MCHCl content assay (%) | 95.0-98.7 | 73.3-86.3 | 80.9-88.6 |

Identified nitrogen mustard degradation products (% w/w)

| | | | |
|---|---|---|---|
| NM Half Mustard | 0.26-0.41 | 2.17-4.45 | 0.96-3.68 |
| NM Transcutol | 0.78-1.12 | 3.55-6.48 | 3.44-6.40 |
| NM Isopropyl Alcohol | 0.57-0.79 | 2.67-4.55 | 2.56-4.64 |
| NM Glycerin | 0.45-0.62 | 2.10-3.15 | 2.51-4.58 |
| NM Propylene Glycol | 0.46-0.57 | 2.07-3.50 | 1.82-4.17 |
| NM Lactoyl Lactate | 0.69-0.93 | 1.97-2.95 | 2.84-4.39 |
| Total identified degradation | 3.3-4.4 | 15.0-24.0 | 14.7-27.4 |
| products (% w/w) | | | |
| Total unidentified degradation | 0.24-0.38 | 0.25-0.37 | 0.72-0.73¹ |
| products (% w/w) | | | |
| Total degradation products | 3.6-4.7 | 15.3-24.4 | 15.4-28.1 |
| (% w/w) | | | |

¹Data at 6-month time point.

TABLE 4

| Identified Nitrogen Mustard | |
|---|---|
| Degradation Product | Structure |
| NM half mustard (DP-1) | 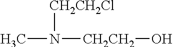 |
| NM Transcutol (DP-9) | 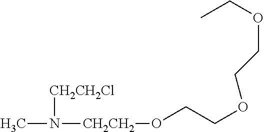 |
| NM Isopropyl Alcohol (DP-7) | 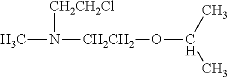 |
| NM Glycerin (DP-5) | 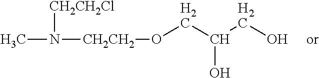 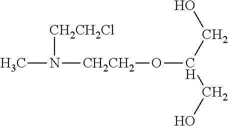 |
| NM Proylene Glycol (DP-15) | 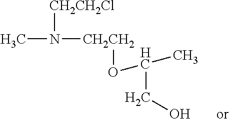 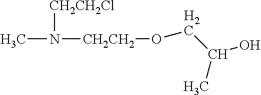 |
| NM Lactoyl Lactate (DP-16) | 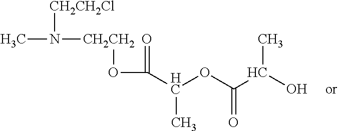 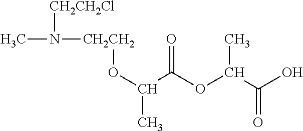 |

We claim:

1. A topical composition comprising: (a) an effective amount of an alkylating agent or a pharmaceutically acceptable salt or solvate thereof; and (b) at least one pharmaceutically acceptable excipient, wherein:
the pharmaceutically acceptable excipient is an alcohol, a ketone, an aldehyde, an ether, an amide, an alkane (linear, branched or cyclic), an alkene (linear, branched or cyclic), an aromatic (fused or non-fused), a dimethyl polysiloxane, a hydroxy ether, a substituted diol, an ethylene glycol derivative, a polyoxylglyceride, a polar aprotic solvent, an alpha-hydroxycarboxylic acid or a salt thereof, a diester of a dibasic acid, a polyethoxylated fatty acid, a PEG-fatty acid diester, a PEG-fatty acid mono-ester or an all-ester mixture, a polyethylene glycol glycerol fatty acid ester, an alcohol-oil transesterification product, a polyglycerized fatty acid, a propylene glycol fatty acid ester, a mixture of a propylene glycol ester and a glycerol ester, a mono- or diglycerides, a sterol or sterol derivative, a polyethylene glycol sorbitan fatty acid ester, a polyethylene glycol alkyl ether, a sugar ester, a polyethylene glycol alkyl phenol, a polyoxyethylene-polyoxypropylene block copolymer, a polyoxyethylene, a sorbitan fatty acid ester, a lower alcohol fatty acid ester, an ionic surfactant, a penetration enhancer, or a thickening agent.

2. The topical composition of claim 1, wherein the pharmaceutically acceptable excipient is a substituted diol.

3. The topical composition of claim 2, wherein the substituted diol is a compound of the formula 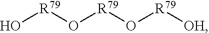
wherein R⁷⁹ is a linear alkyl group having 1-12 carbon atoms, or a branched alkyl group having 2-12 carbon atoms.

4. The topical composition of claim 1, wherein the pharmaceutically acceptable excipient is a hydroxy ether.

5. The topical composition of claim 4, wherein the hydroxy ether is a compound of the formula 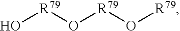
wherein R⁷⁹ is a linear alkyl group having 1-12 carbon atoms, or a branched alkyl group having 2-12 carbon atoms.

6. The topical composition of claim 1, wherein the pharmaceutically acceptable excipient is an ethylene glycol derivative.

7. The topical composition of claim 1, wherein the ethylene glycol derivative is butylene glycol, dipropylene glycol, hexylene glycol, ethyl hexanediol, propylene glycol monolaurate, propylene glycol monostearate, propylene glycol palmitostearate, propylene glycol ricinoleate, glyceryl acetate, glyceryl citrate, glyceryl isostearate, glyceryl laurate, glyceryl monostearate, glyceryl oleate, glyceryl palmitate, glyceryl ricinoleate, glyceryl stearate-laureth-23, glyceryl stearate/PEG-100 stearate, or 1,2,6-hexanetriol.

8. The topical composition of any one of claims 1 to 7, wherein the alkylating agent is a nitrogen mustard.

9. The topical composition of claim 8, wherein the nitrogen mustard is a compound of the following Structure (VII), (VIII), (IX), (X), (XII), (XIII), (XIV), (XV), (XVI), (XVII), (XVIII), or (XIX): 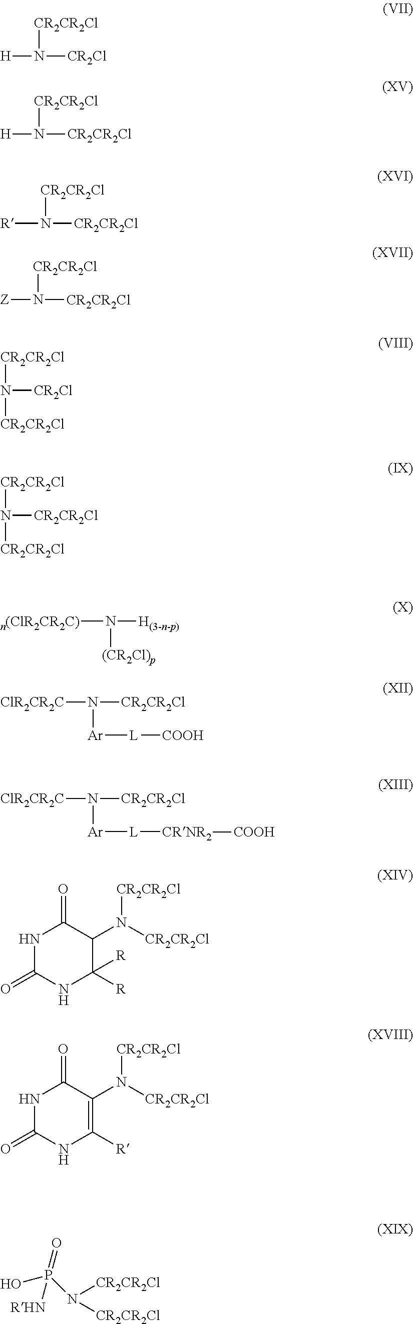
wherein:
each R and R′ is independently selected from the group consisting of H, a linear alkyl group having 1-6 carbon atoms, a branched alkyl group having 2-12 carbon atoms, a cycloalkyl group having 3-17 carbon atoms, a fluorinated linear alkyl group having 2-12 carbon atoms, a fluorinated branched alkyl group having 2-12 carbon atoms, a fluorinated cycloalkyl group having 3-17 carbon atoms, an aryl group, an aralkyl group, an alkaryl group, a cycloalkyl group, a bicycloalkyl group, an alkenyl group, an alkalkenyl group, an alkenylalkyl group, an alkynyl group, an alkalkynyl group, an alkynylalkyl group, a trifluoropropyl group, a cyanopropyl group, an acryloyl group, an arylacryloyl group, an acryloylaryl group, an alkylacyl group, an arylacyl group, an alkylenylacyl group, and an alkynylacyl group, wherein any two R in the same molecule are optionally linked to form a three- to eight-membered cyclic group;
Z is a linear alkyl group having 1-6 carbon atoms;
each L is independently a linking group selected from the group consisting of linear or branched alkylene having 1 to 7 carbon atoms, cycloalkylene having 3 to 17 carbon atoms, alkylcycloalkylene having 4 to 20 carbon atoms, a cycloalkylalkylene having 4 to 20 carbon atoms, an arylene, having 4 to 30 carbon atoms, an alkylarylene, having 4 to 30 carbon atoms, an arylalkylene, having 4 to 30 carbon atoms, and combinations thereof;
each Ar is independently a bifunctional aromatic linking group wherein each Ar is selected from the group consisting of arylene, substituted arylene and heteroarylene;
n is 1, 2, or 3;
p is 0, 1, or 2; and
n+p≦3.

10. The composition of claim 8, wherein the nitrogen mustard is a compound of the following Structure (XVII) 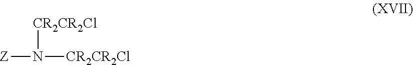
wherein Z is a linear alkyl group having 1-6 carbon atoms and each R is independently hydrogen or a linear alkyl group having 1-6 carbon atoms.

11. The composition of claim 10, wherein Z is methyl or ethyl.

12. The composition of claim 8, wherein the nitrogen mustard is bis-(2-chloroethyl) ethylamine, bis-(2-chloroethyl)methylamine, or tris-(2-chloroethyl)amine.

13. The composition of claim 8, wherein the nitrogen mustard is

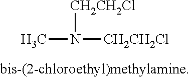

14. The composition of any one of claims 1 to 13, wherein the composition has a viscosity of about 5,000 cps to about 50,000 cps.

15. The composition of any one of claims 1 to 13, wherein the composition has a viscosity of about 15,000 cps to about 40,000 cps.

16. The composition of any one of claims 1 to 13, wherein the composition has a viscosity of about 20,000 cps to about 35,000 cps.

17. The composition of any one of claims 1 to 16, wherein at least about 90% of the alkylating agent or pharmaceutically acceptable salt or solvate thereof is present in the composition after storage for at least about 3, about 6, or about 12 months at a temperature of about −20° C. or higher.

18. The composition of claim 17, wherein at least about 90% of the alkylating agent or pharmaceutically acceptable salt or solvate thereof is present in the composition after storage for at least about 3, about 6, or about 12 months at a temperature of about 2° C. or higher.

19. The composition of claim 17, wherein at least about 90% of the alkylating agent or pharmaceutically acceptable salt or solvate thereof is present in the composition after storage for at least about 3, about 6, or about 12 months at a temperature of about 2° C. to about 8° C.

20. The composition of claim 17, wherein at least about 90% of the alkylating agent or pharmaceutically acceptable salt or solvate thereof is present in the composition after storage for at least about 3, about 6, or about 12 months at a temperature of about −20° C. to about −10° C.

21. The composition of any one of claims 1 to 16, wherein at least about 90% of the alkylating agent or pharmaceutically acceptable salt or solvate thereof is present in the composition after storage for at least about 1, about 2, about 3, about 6, or about 12, months at a temperature of about 15° C. to about 30° C.

22. The composition of any one of claims 8 to 13, wherein the composition contains less than about 10% by weight of nitrogen mustard degradation product after storage for at least about 3, about 6, or about 12 months at a temperature of about −20° C. or higher.

23. The composition of claim 22, wherein the composition contains less than about 10% by weight of nitrogen mustard degradation product after storage for at least about 3, about 6, or about 12 months at a temperature of about 2° C. or higher.

24. The composition of claim 22, wherein the composition contains less than about 10% by weight of nitrogen mustard degradation product after storage for at least about 3, about 6, or about 12 months at a temperature of about 2° C. to about 8° C.

25. The composition of claim 22, wherein the composition contains less than about 10% by weight of nitrogen mustard degradation product after storage for at least about 3, about 6, or about 12 months at a temperature of about −20° C. to about −10° C.

26. The composition of any one of claims 8 to 13, wherein the composition contains less than about 10% by weight of nitrogen mustard degradation product after storage for at least about 1, about 2, about 3, about 6, or about 12 months at a temperature of about 15° C. to about 30° C.

27. The composition of any one of claims 22 to 26, wherein the nitrogen mustard degradation product is

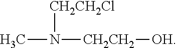

28. The composition of any one of claims 22 to 26, wherein the nitrogen mustard degradation product is:

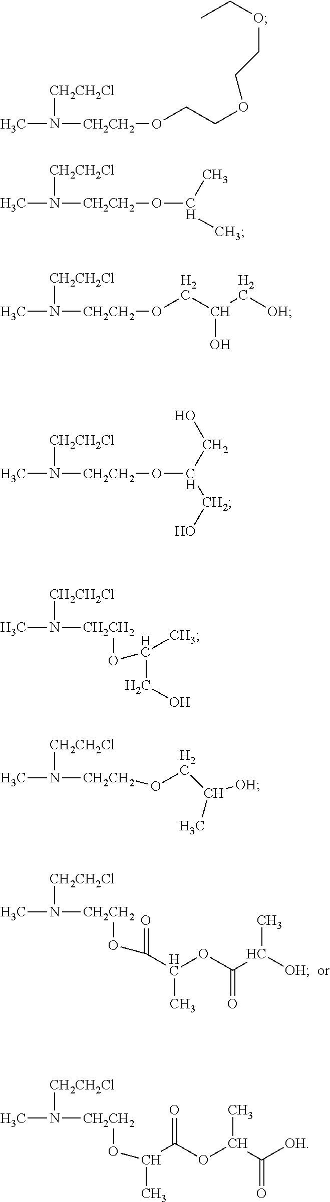

29. The composition of any one of claims 1 to 28 further comprising an effective amount of a steroid.

30. The composition of claim 29, wherein the steroid is betamethasone, clobetasol, fluocinonide, halobetasol, desoximetasone, diflorasone, halocinonide, triamcinolone, amcinonide, flurandrenolide, fluticasone, mometasone, desonide, hydrocortisone, prednicarbate, alclometasone, clocortolone, amcinonide, fluocinonlone, clobetasone, desonide, or bexarotine.

31. A method for treating a skin disorder comprising topically applying to a subject in need thereof a composition of any one of claims 1 to 30.

32. The method of claim 31, wherein the skin disorder is psoriasis, eczema, actinic keratosis, lupus, sarcoidosis, alopecia, cutaneous T-Cell lymphoma, mycosis fungoides, lymphoreticular neoplasia, pleural or peritoneal effusions, cutaneous B-cell lymphoma, pseudolymphoma of the skin, squamous cell carcinoma, basal cell carcinoma, bronchogenic carcinoma, malignant melanoma, lymphosarcoma, chronic lymphocytic leukemia, polycythemia vera, lymphomatoid papulosis, Mucha-Habberman's disease, or vitiligo.

33. The method of claim 31, wherein the skin disorder is a T-cell mediated skin disorder.

34. The method of claim 33, wherein the T-cell mediated skin disorder is psoriasis, actinic keratosis, cutaneous T-cell lymphoma, cutaneous B-cell lymphoma, mycosis fungoides, alopecia, alopecia greata, or vitiligo.

35. The method of claim 31, wherein the skin disorder is mycosis fungoides.

36. The method of claim 31, wherein the response rate in a group of human patients is greater than about 60% after at least six months of treatment.

37. The method of claim 31, wherein the response rate in a group of human patients is greater than about 55% upon two months of treatment.

38. The method of claim 31, wherein the response rate in an intent-to-treat group of human patients is greater than about 50%.

39. The method of claim 31, wherein the time to achieve a response rate of 50% in a group of human patients is about 40 weeks or less.

40. The method of claim 39, wherein the time to achieve a response rate of 50% in a group of human patients is about 30 weeks or less.

41. The topical composition of claim 3, wherein each R⁷⁹ group of the substituted diol is independently a group of the formula: 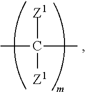
wherein each Z¹ is independently H, linear C₁-C₁₂ alkyl, branched C₃-C₁₂ alkyl, cyclic C₃-C₁₂ alkyl, linear C₂-C₁₂ alkenyl, branched C₃-C₁₂ alkenyl, cyclic C₅-C₁₂ alkenyl, linear C₂-C₁₂ alkynyl, branched C₄-C₁₂ alkynyl, cyclic C₈-C₁₂ alkynyl, aryl, heteroaryl, or heterocyclyl, any of which is optionally-substituted with any number of halogens, or two vicinal or geminal Z¹ groups can together form a carbocyclic or heterocyclic ring with the carbon atom(s) to which the Z¹ groups are attached; and m is 1-12.

42. The topical composition of claim 2, wherein the substituted diol is a compound of the formula: 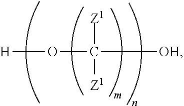
wherein each Z¹ is independently H, linear C₁-C₁₂ alkyl, branched C₃-C₁₂ alkyl, cyclic C₃-C₁₂ alkyl, linear C₂-C₁₂ alkenyl, branched C₃-C₁₂ alkenyl, cyclic C₅-C₁₂ alkenyl, linear C₂-C₁₂ alkynyl, branched C₄-C₁₂ alkynyl, cyclic C₈-C₁₂ alkynyl, aryl, aralkyl, alkaryl, heteroaryl, hetereoarylalkyl, alkheteroaryl, heterocyclyl, heterocyclylalkyl, or alkheterocyclyl, any of which is optionally-substituted with any number of halogens, or two vicinal or geminal Z¹ groups can together form a carbocyclic or heterocyclic ring with the carbon atom(s) to which the Z¹ groups are attached; each m is independently 1-12; and n is 3-8.

43. The topical composition of claim 42, wherein the diol is a compound of the formula:

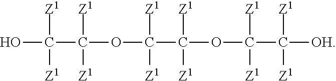

44. The topical composition of claim 5, wherein each R⁷⁹ group of the hydroxy ether is independently a group of the formula: 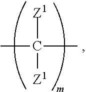
wherein each Z¹ is independently H, linear C₁-C₁₂ alkyl, branched C₃-C₁₂ alkyl, cyclic C₃-C₁₂ alkyl, linear C₂-C₁₂ alkenyl, branched C₃-C₁₂ alkenyl, cyclic C₅-C₁₂ alkenyl, linear C₂-C₁₂ alkynyl, branched C₄-C₁₂ alkynyl, cyclic C₈-C₁₂ alkynyl, aryl, heteroaryl, or heterocyclyl, any of which is optionally-substituted with any number of halogens, or two vicinal or geminal Z¹ groups can together form a carbocyclic or heterocyclic ring with the carbon atom(s) to which the Z¹ groups are attached; and m is 1-12.

45. The topical composition of claim 4, wherein the hydroxy ether is a compound of the formula: 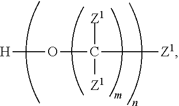
wherein each Z¹ is independently H, linear C₁-C₁₂ alkyl, branched C₃-C₁₂ alkyl, cyclic C₃-C₁₂ alkyl, linear C₂-C₁₂ alkenyl, branched C₃-C₁₂ alkenyl, cyclic C₅-C₁₂ alkenyl, linear C₂-C₁₂ alkynyl, branched C₄-C₁₂ alkynyl, cyclic C₈-C₁₂ alkynyl, aryl, aralkyl, alkaryl, heteroaryl, hetereoarylalkyl, alkheteroaryl, heterocyclyl, heterocyclylalkyl, or alkheterocyclyl, any of which is optionally-substituted with any number of halogens, or two vicinal or geminal Z¹ groups can together form a carbocyclic or heterocyclic ring with the carbon atom(s) to which the Z¹ groups are attached; each m is independently 1-12, and n is 3-8.

46. The topical composition of claim 45, the hydroxy ether is a compound of the formula: [chemical structure]
